SEQUENCE LISTING (101 sequences)

SEQ ID NO: 1 - protein, 119 aa, synthetic construct
  source 1..119: note = m912 VH sequence
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 2 - protein, 107 aa, synthetic construct [sequence omitted]

SEQ ID NO: 3 - DNA, 37 bp, synthetic construct
  source 1..37: mol_type = other DNA; note = HCDR1 HP1-1 Forward primer
tccgtcnnsnnsnnsnnstactactggagctggatcc

SEQ ID NO: 4 - DNA, 22 bp, synthetic construct
  source 1..22: mol_type = other DNA; note = HCDR1 HP1-1 Reverse primer
gccaccagagacagtgcaggtg

SEQ ID NO: 5 - DNA, 37 bp, synthetic construct
  source 1..37: mol_type = other DNA; note = HCDR1 HP1-2 Forward primer
gtcagcnnsnnsnnsnnstactggagctggatccggc

SEQ ID NO: 6 - DNA, 22 bp, synthetic construct
  source 1..22: mol_type = other DNA; note = HCDR1 HP1-2 Reverse primer
ggagccaccagagacagtgcag

SEQ ID NO: 7 - DNA, 37 bp, synthetic construct
  source 1..37: mol_type = other DNA; note = HCDR1 HP1-3 Forward primer
agcagtnnsnnsnnsnnstggagctggatccggcagc

SEQ ID NO: 8 - DNA, 22 bp, synthetic construct
  source 1..22: mol_type = other DNA; note = HCDR1 HP1-3 Reverse primer
gacggagccaccagagacagtg

SEQ ID NO: 9 - DNA, 41 bp, synthetic construct
  source 1..41: mol_type = other DNA; note = HCDR2 HP2-1 Forward primer
tattacnnsnnsnnsnnsnnstacaacccctccctcaagag

SEQ ID NO: 10 - DNA, 21 bp, synthetic construct
  source 1..21: mol_type = other DNA; note = HCDR2 HP2-1 Reverse primer
gatatacccaatccactccag

SEQ ID NO: 11 - DNA, 38 bp, synthetic construct
  source 1..38: mol_type = other DNA; note = HCDR3 HP3-1 Forward primer
gcgagannsnnsnnsnnsggggcttttgatatctgggg

SEQ ID NO: 12 - DNA, 21 bp, synthetic construct
  source 1..21: mol_type = other DNA; note = HCDR3 HP3-1 Reverse primer
acagtaatacacggccgtgtc

SEQ ID NO: 13 - DNA, 38 bp, synthetic construct
  source 1..38: mol_type = other DNA; note = HCDR3 HP3-2 Forward primer
agagagnnsnnsnnsnnsgcttttgatatctggggcca

SEQ ID NO: 14 - DNA, 20 bp, synthetic construct
  source 1..20: mol_type = other DNA; note = HCDR3 HP3-2 Reverse primer
cgcacagtaatacacggccg

SEQ ID NO: 15 - DNA, 38 bp, synthetic construct
  source 1..38: mol_type = other DNA; note = HCDR3 HP3-3 Forward primer
gaggggnnsnnsnnsnnstttgatatctggggccaagg

SEQ ID NO: 16 - DNA, 20 bp, synthetic construct
  source 1..20: mol_type = other DNA; note = HCDR3 HP3-3 Reverse primer
tctcgcacagtaatacacgg

SEQ ID NO: 17 - DNA, 44 bp, synthetic construct
  source 1..44: mol_type = other DNA; note = HCDR3 HP3-4 Forward primer
gcgagagasnnsnnsnnsnnsnnstttgatatctggggccaagg

SEQ ID NO: 18 - DNA, 21 bp, synthetic construct
  source 1..21: mol_type = other DNA; note = HCDR3 HP3-4 Reverse primer
acagtaatacacggccgtgtc

SEQ ID NO: 19 - DNA, 40 bp, synthetic construct
  source 1..40: mol_type = other DNA; note = LCDR1 LP1-1 Forward primer
cagagcnnsnnsnnsnnsnnsaattggtatcagcagaaac

SEQ ID NO: 20 - DNA, 22 bp, synthetic construct
  source 1..22: mol_type = other DNA; note = LCDR1 LP1-1 Reverse primer
acttgcccggcaagtgatggtg

SEQ ID NO: 21 - DNA, 37 bp, synthetic construct
  source 1..37: mol_type = other DNA; note = LCDR2 LP2-1 Forward primer
ctgatcnnsnnsnnsnnsagtttgcaaagtggggtcc

SEQ ID NO: 22 - DNA, 21 bp, synthetic construct
  source 1..21: mol_type = other DNA; note = LCDR2 LP2-1 Reverse primer
gagcttaggggctttccctgg

SEQ ID NO: 23 - DNA, 38 bp, synthetic construct
  source 1..38: mol_type = other DNA; note = LCDR2 LP2-2 Forward primer
gcatccnnsnnsnnsnnsggggtcccatcaaggttcag

SEQ ID NO: 24 - DNA, 21 bp, synthetic construct
  source 1..21: mol_type = other DNA; note = LCDR2 LP2-2 Reverse primer
agcatagatcaggagcttagg

SEQ ID NO: 25 - DNA, 39 bp, synthetic construct
  source 1..39: mol_type = other DNA; note = LCDR3 LP3-1 Forward primer
tactgtnnsnnsnnsnnsagtaccccgctcactttcggc

SEQ ID NO: 26 - DNA, 20 bp, synthetic construct
  source 1..20: mol_type = other DNA; note = LCDR3 LP3-1 Reverse primer
gtaagttgcaaaatcttcag

SEQ ID NO: 27 - DNA, 39 bp, synthetic construct
  source 1..39: mol_type = other DNA; note = LCDR3 LP3-2 Forward primer
caacagnnsnnsnnsnnsccgctcactttcggcggaggg

SEQ ID NO: 28 - DNA, 21 bp, synthetic construct
  source 1..21: mol_type = other DNA; note = LCDR3 LP3-2 Reverse primer
acagtagtaagttgcaaaatc

SEQ ID NO: 29 - DNA, 39 bp, synthetic construct
  source 1..39: mol_type = other DNA; note = LCDR3 LP3-3 Forward primer
agttacnnsnnsnnsnnsactttcggcggagggaccaag

SEQ ID NO: 30 - DNA, 21 bp, synthetic construct
  source 1..21: mol_type = other DNA; note = LCDR3 LP3-3 Reverse primer
ctgttgacagtagtaagttgc

SEQ ID NO: 31 - DNA, 39 bp, synthetic construct
  source 1..39: mol_type = other DNA; note = LCDR3 LP3-4 Forward primer
agtaccnnsnnsnnsnnsggcggagggaccaaggtggag

SEQ ID NO: 32 - DNA, 23 bp, synthetic construct
  source 1..23: mol_type = other DNA; note = LCDR3 LP3-4 Reverse primer
gtaactctgttgacagtagtaag

SEQ ID NO: 33 - DNA, 45 bp, synthetic construct
  source 1..45: mol_type = other DNA; note = LCDR3 LP3-5 Forward primer
caacagnnstwcnnsnnsnnsnnsactttcggcggagggaccaag

SEQ ID NO: 34 - DNA, 21 bp, synthetic construct
  source 1..21: mol_type = other DNA; note = LCDR3 LP3-5 Reverse primer
acagtagtaagttgcaaaatc

SEQ ID NO: 35 - protein, 6 aa, synthetic construct
  source 1..6: note = HCDR1-2
SSYHDL

SEQ ID NO: 36 - protein, 6 aa, synthetic construct
  source 1..6: note = HCDR1-3
SSRDDL

SEQ ID NO: 37 - protein, 6 aa, synthetic construct
  source 1..6: note = HCDR1-9
SSFTDT

SEQ ID NO: 38 - protein, 6 aa, synthetic construct
  source 1..6: note = HCDR1-14
SSYYST

SEQ ID NO: 39 - protein, 6 aa, synthetic construct
  source 1..6: note = HCDR3 HP1-A13
DDVLGA

SEQ ID NO: 40 - protein, 6 aa, synthetic construct
  source 1..6: note = HCDR3 HP4-44
EDVLSG

SEQ ID NO: 41 - protein, 6 aa, synthetic construct
  source 1..6: note = HCDR3-6
EDALTG

SEQ ID NO: 42 - protein, 6 aa, synthetic construct
  source 1..6: note = HCDR3-7
DFVLSG

SEQ ID NO: 43 - protein, 6 aa, synthetic construct
  source 1..6: note = HCDR3-8
DNVFTG

SEQ ID NO: 44 - protein, 5 aa, synthetic construct
  source 1..5: note = LCDR1-6
SRYYV

SEQ ID NO: 45 - protein, 6 aa, synthetic construct
  source 1..6: note = LCDR3-2
SYDASY

SEQ ID NO: 46 - protein, 6 aa, synthetic construct
  source 1..6: note = LCDR3 LP2
STDAFY

SEQ ID NO: 47 - protein, 6 aa, synthetic construct
  source 1..6: note = LCDR3 LP5-1
PYDASY

SEQ ID NO: 48 - protein, 119 aa, synthetic construct
  source 1..119: note = HP1-A13 VH sequence
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARDDVLGAFDIWGQGTMVTVSS

SEQ ID NO: 49 - protein, 119 aa, synthetic construct
  source 1..119: note = HP4-44 VH sequence
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREDVLSGFDIWGQGTMVTVSS

SEQ ID NO: 50 - protein, 107 aa, synthetic construct
  source 1..107: note = HP4-44 VL sequence
DIQMTQSPSSLSASVGDRVTITCRASQSISSYLNWYQQKPGKAPKLLIYAASSLQSGVPSRFSGSGSGTDFTLTISSLQPEDFATYYCQQSYDAFYTFGGGTKVEIK

SEQ ID NO: 51 - protein, 119 aa, synthetic construct
  source 1..119: note = MSLN-4, MSLN-28, VH sequence
QVQLQESGPGLVKPSETLSLTCTVSGGSVSSYYSTWSWIRQPPGKGLEWIGYIYYSGSTNYNPSLKSRVTISVDTSKNQFSLKLSSVTAADTAVYYCAREDALTGFDIWGQGTMVTVSS

SEQ ID NO: 52 - protein, 107 aa, synthetic construct
  source 1..107: note = MSLN-4 VL sequence
DIQMTQSPSSLSASVGDRVTITCRASQSISSYLNWYQQKPGKAPKLLIYAASSLQSGVPSRFSGSGSGTDFTLTISSLQPEDFATYYCQQSYDASYTFGGGTKVEIK

SEQ ID NO: 53 - protein, 119 aa, synthetic construct
  source 1..119: note = MSLN-21,MSLN-33,MSLN-45 VH sequence
QVQLQESGPGLVKPSETLSLTCTVSGGSVSSYHDLWSWIRQPPGKGLEWIGYIYYSGSTNYNPSLKSRVTISVDTSKNQFSLKLSSVTAADTAVYYCARDNVFTGFDIWGQGTMVTVSS

SEQ ID NO: 54 - protein, 107 aa, synthetic construct
  source 1..107: note = MSLN-21 VL sequence
DIQMTQSPSSLSASVGDRVTITCRASQSSRYYVNWYQQKPGKAPKLLIYAASSLQSGVPSRFSGSGSGTDFTLTISSLQPEDFATYYCQQSYDASYTFGGGTKVEIK

SEQ ID NO: 55 - protein, 107 aa, synthetic construct
  source 1..107: note = MSLN-28, MSLN-30, MSLN-31, MSLN-32, MSLN-33 VL sequence
DIQMTQSPSSLSASVGDRVTITCRASQSISSYLNWYQQKPGKAPKLLIYAASSLQSGVPSRFSGSGSGTDFTLTISSLQPEDFATYYCQQPYDASYTFGGGTKVEIK

SEQ ID NO: 56 - protein, 119 aa, synthetic construct
  source 1..119: note = MSLN-30, MSLN-42 VH sequence
QVQLQESGPGLVKPSETLSLTCTVSGGSVSSRDDLWSWIRQPPGKGLEWIGYIYYSGSTNYNPSLKSRVTISVDTSKNQFSLKLSSVTAADTAVYYCARDFVLSGFDIWGQGTMVTVSS

SEQ ID NO: 57 - protein, 115 aa, synthetic construct
  source 1..115: note = MSLN-31 VH sequence
QVQLQESGPGLVKPSETLSLTCTVSGGSVSSFTDTWSWIRQPPGKGLEWIGYIYYSGSTNYNPSLKSRVTISVDTSKNQFSLKLSSVTAADTAVYYCARDFVLSGFDIWGQGTMV

SEQ ID NO: 58 - protein, 119 aa, synthetic construct
  source 1..119: note = MSLN-32 VH sequence
QVQLQESGPGLVKPSETLSLTCTVSGGSVSSYYSTWSWIRQPPGKGLEWIGYIYYSGSTNYNPSLKSRVTISVDTSKNQFSLKLSSVTAADTAVYYCARDFVLSGFDIWGQGTMVTVSS

SEQ ID NO: 59 - protein, 119 aa, synthetic construct
  source 1..119: note = MSLN-39 VH sequence
QVQLQESGPGLVKPSETLSLTCTVSGGSVSSFTDTWSWIRQPPGKGLEWIGYIYYSGSTNYNPSLKSRVTISVDTSKNQFSLKLSSVTAADTAVYYCAREDALTGFDIWGQGTMVTVSS

SEQ ID NO: 60 - protein, 107 aa, synthetic construct; identical to SEQ ID NO: 54
  source 1..107: note = MSLN-39, MSLN-40, MSLN-42, MSLN-45 VL sequence

SEQ ID NO: 61 - protein, 6 aa, synthetic construct
  source 1..6: note = HCDR1
SSGSYY

SEQ ID NO: 62 - protein, 9 aa, synthetic construct
  source 1..9: note = HCDR2
YIYYSGSTN

SEQ ID NO: 63 - protein, 6 aa, synthetic construct
  source 1..6: note = HCDR3
EGKNGA

SEQ ID NO: 64 - protein, 8 aa, synthetic construct
  source 1..8: note = LCDR1
QSISSYLN

SEQ ID NO: 65 - protein, 8 aa, synthetic construct
  source 1..8: note = LCDR2
YAASSLQS

SEQ ID NO: 66 - protein, 8 aa, synthetic construct
  source 1..8: note = LCDR3
QQSYSTPL

SEQ ID NO: 67 - protein, 4 aa, synthetic construct
  source 1..4: note = HCDR1 HP1-1 Target sequence
SSGS

SEQ ID NO: 68 - protein, 4 aa, synthetic construct
  source 1..4: note = HCDR1 HP1-2 Target sequence
SGSY

SEQ ID NO: 69 - protein, 4 aa, synthetic construct
  source 1..4: note = HCDR1 HP1-3 Target sequence
GSYY

SEQ ID NO: 70 - protein, 5 aa, synthetic construct
  source 1..5: note = HCDR2 HP2-1 Target sequence
SGSTN

SEQ ID NO: 71 - protein, 4 aa, synthetic construct
  source 1..4: note = HCDR3 HP3-1 Target sequence
EGKN

SEQ ID NO: 72 - protein, 4 aa, synthetic construct
  source 1..4: note = HCDR3 HP3-2 Target sequence
GKNG

SEQ ID NO: 73 - protein, 4 aa, synthetic construct
  source 1..4: note = HCDR3 HP3-3 Target sequence
KNGA

SEQ ID NO: 74 - protein, 6 aa, synthetic construct
  source 1..6: note = HCDR3 HP3-4 Target sequence
EGKNGA

SEQ ID NO: 75 - protein, 5 aa, synthetic construct
  source 1..5: note = LCDR1 LP1-1 Target sequence
ISSYL

SEQ ID NO: 76 - protein, 4 aa, synthetic construct
  source 1..4: note = LCDR2 LP2-1 Target sequence
YAAS

SEQ ID NO: 77 - protein, 4 aa, synthetic construct
  source 1..4: note = LCDR2 LP2-2 Target sequence
SLQS

SEQ ID NO: 78 - protein, 4 aa, synthetic construct
  source 1..4: note = LCDR3 LP3-1 Target sequence
QQSY

SEQ ID NO: 79 - protein, 4 aa, synthetic construct
  source 1..4: note = LCDR3 LP3-2 Target sequence
SYST

SEQ ID NO: 80 - protein, 4 aa, synthetic construct
  source 1..4: note = LCDR3 LP3-3 Target sequence
STPL

SEQ ID NO: 81 - protein, 4 aa, synthetic construct
  source 1..4: note = LCDR3 LP3-4 Target sequence
PLTF

SEQ ID NO: 82 - protein, 6 aa, synthetic construct
  source 1..6: note = LCDR3 LP3-5 Target sequence
SYSTPL

SEQ ID NO: 83-101 - skipped (19 entries)

(19) United States
(12) Patent Application Publication
(10) Pub. No.: US 2026/0209319 A1
(43) Pub. Date: Jul. 23, 2026

(54) ANTI-MESOTHELIN BISPECIFIC ANTIBODIES AND METHODS OF USE

(71) Applicant: JECHO LABORATORIES, INC., Frederick, MD (US)

(72) Inventors: Jianwei Zhu, Frederick, MD (US); Shusheng Wang, Ellicott City, MD (US); Zhangyi Song, Rockville, MD (US); Haiqiu Huang, North Bethesda, MD (US); Kaiyong Yang, Potomac, MD (US); Xiaodong Xiao, Frederick, MD (US); Yueqing Xie, Clarksburg, MD (US); Hua Jiang, Clarksburg, MD (US)

(21) Appl. No.: 19/134,389

(22) PCT Filed: Dec. 1, 2023
(86) PCT No.: PCT/US2023/082061

§ 371 Date: May 30, 2025

Related U.S. Application Data
(60) Provisional application No. 63/429,821, filed on Dec. 2, 2022.

(51) Int. Cl. C07K 16/18; A61K 39/00; A61K 47/68; A61P 35/00; C07K 16/28

(52) U.S. Cl. CPC C07K 16/18; A61K 47/6849; A61P 35/00; C07K 16/2809; A61K 2039/505; C07K 2317/31; C07K 2317/565; C07K 2317/622; C07K 2317/92

(57) ABSTRACT

The disclosure provides amino acid sequences (e.g., antibodies and bispecific antibodies) that can bind mesothelin (MSLN). The disclosure relates to bispecific antibodies and antigen binding fragments thereof, conjugates, including immunoconjugates, having specific binding to mesothelin, and related compositions and formulations. The disclosure also relates to methods for using the mesothelin bispecific antibodies and antigen-binding fragments thereof, in the treatment of mesothelin-related diseases, disorders, and conditions including cell proliferative diseases such as cancer.

### CROSS REFERENCE TO RELATED APPLICATIONS

This application is related to, and claims priority to, U.S. Provisional Patent Application Ser. No. 63/429,821, filed Dec. 2, 2022, and which is herein incorporated by reference in its entirety.

### BACKGROUND

Mesothelin is a GPI-anchored glycoprotein that normally expresses in pleural spaces and other tissues commonly related to reproduction. It has been demonstrated to be a tumor-associated protein with overexpression in various types of cancer including mesothelioma, pancreatic adenocarcinoma, ovarian cancer, non-small cell lung cancer (NSCLC), and breast cancer. Mesothelin is considered to be a candidate for targeted cancer therapy [1]. Several anti-mesothelin antibodies have been developed in various formats for preclinical and clinical evaluations [2-5]. These antibodies have been derived either from hybridomas or from fully humanized phage libraries and have been tested in formats including naked antibodies, antibody drug conjugates, immunotoxins, and CAR-T. Early clinical investigations with mesothelin-targeted CAR-T cells reported good results [6]; however continued development of additional therapeutics (e.g., cancer therapies) that target mesothelin is needed.

An antibody, m912 [6], was used in the CAR-T platform and is derived from a fully human Fab library [7]. It has nearly 100% germline sequence, but its affinity is poor (no better than 20 nM as reported [7]). Nevertheless, this low-affinity antibody exhibited good phase I clinical results in a CAR-T format [6]. CAR-T is known to tolerate antibodies with relatively poor affinity for reasons that are not fully understood. Because of this feature of CAR-T, the affinity of m912 may not be good enough when incorporated in other formats such as ADC or bispecific antibodies. As such further development around this antibody and other antibodies and proteins that bind specifically to mesothelin is needed.

Described herein are specific binding proteins, antibodies, bispecific antibodies, monoclonal antibodies, and antigen-binding fragments and domains thereof that can bind mesothelin and can be used in methods for detection, treatment, and prophylaxis of mesothelin-related disorders, diseases, and associated clinical symptoms, including cancer.

### SUMMARY OF THE DISCLOSURE

The disclosure generally provides an antibody (i.e., isolated antibodies, bispecific antibodies, and antigen-binding fragments thereof), that bind to mesothelin (MSLN). In some aspects, the disclosure provides an antibody, bispecific antibody, isolated antibodies, and antigen-binding fragments thereof that exhibit specific binding to an epitope of mesothelin. In some embodiments, the antibody, bispecific antibody, or antigen-binding fragment thereof can comprise one or more heavy chain variable regions, one or more light chain variable regions, one or more CDR1 of a light and/or heavy chain variable region, one or more CDR2 of a light and/or heavy chain variable region, one or more CDR3 of a light and/or heavy chain variable region, or any combinations thereof, according to the aspects and embodiments provided by the disclosure.

In embodiments, the antibody, bispecific antibody, or antigen-binding fragment thereof binds to an epitope within the sequence of a human mesothelin protein sequence. In embodiments, the disclosure provides a bispecific antibody that binds human mesothelin and a targeted sequence other than mesothelin.

In some aspects, the disclosure relates to an isolated antibody, bispecific antibody, or antigen-binding fragment thereof that binds to mesothelin (MSLN) protein, the antibody, bispecific antibody, or antigen-binding fragment thereof comprising: (i) a heavy chain variable region comprising: a CDR1, a CDR2, and a CDR3 of the amino acid sequences as disclosed herein; and a light chain variable region comprising: a CDR1, a CDR2, and a CDR3 of the amino acid sequences as disclosed herein.

In some embodiments of this aspect, the disclosure provides an isolated antibody, bispecific antibody, or antigen-binding fragment thereof, comprising: (i) a heavy chain variable region having an amino acid sequence as disclosed herein; and a light chain variable region having an amino acid sequence as disclosed herein.

In some embodiments of the above aspects, the disclosure provides an antigen-binding fragment comprising a single-chain Fv (scFv), a single chain Fv-Fc (scFv-Fc), a single-chain antibody, a single domain antibody, a Fab fragment, or a F(ab′)2 fragment.

In some embodiments of the above aspects, the disclosure provides an antibody or bispecific antibody comprising an IgG, an IgM, an IgA, an IgE, an IgD, or an IgY isotype.

In some embodiments of the above aspects, the disclosure provides a monoclonal antibody (mAb) or a monoclonal, bispecific antibody (mbsAb).

In some embodiments of the above aspects, the disclosure provides an antibody, bispecific antibody, or an antigen-binding fragment that is conjugated to a moiety. In some embodiments, the conjugated moiety comprises a therapeutic agent, an active agent, a solid support, an affinity agent, or a detectable label. In some further embodiments, the moiety comprises a cytotoxin. In some other embodiments, the moiety comprises an anti-cancer agent.

In some embodiments of the above aspects, the antibody, bispecific antibody, or an antigen-binding fragment thereof binds to mesothelin with a K_{D} of from about 1.0 pM to 200 nM.

In some embodiments of the above aspects, the antibody, bispecific antibody, or antigen-binding fragment thereof inhibits cell proliferation with an IC50 of from about 0.01-250 nM.

In another aspect, the disclosure provides a bispecific antibody that comprises an antibody, bispecific antibody, or antigen-binding fragment thereof in accordance with any of the above aspects and embodiments, and an antibody, bispecific antibody, or antigen-binding fragment thereof that specifically binds to an antigen that does not comprise a mesothelin epitope. In some further embodiments, the bispecific antibody comprises a mesothelin antibody and a therapeutic antibody. In further embodiments, the bispecific antibody comprises an antibody, bispecific antibody, or antigen-binding fragment thereof that specifically binds to an antigen that does not comprise a mesothelin epitope and induces an immune response. In yet further embodiments, the bispecific antibody comprises a mesothelin antibody, bispecific antibody, or fragment thereof in accordance with the disclosure and antibody, bispecific antibody, or antigen-binding fragment thereof that specifically binds to an antigen other than mesothelin, comprising CD2, CD3, CD11a CD20, CD25 (IL2R), CD33, CD52, EGFR, VEGF, Integrin-alpha 3, GPIIb/IIIar, Protein F, TNF-alpha, TNF-beta, HER2/Neu, C5, or IgE.

In any of the above aspects and embodiments, the isolated antibody, bispecific antibody, or antigen-binding fragment thereof can be conjugated to a solid support, an affinity agent, or a detectable agent.

In another aspect, the disclosure provides a pharmaceutical composition comprising the isolated antibody, bispecific antibody, or antigen-binding fragment thereof according to any of the above aspect and embodiments and a pharmaceutically acceptable carrier.

In another aspect, the disclosure provides a kit comprising the pharmaceutical composition or the antibody, bispecific antibody, or antigen-binding fragment thereof according to any of the above aspects and embodiments, optional reagents, and instructions for use.

In another aspect, the disclosure provides a method for treating cancer in a subject in need thereof, the method comprising administering to the subject an effective amount of an antibody, bispecific antibody, or antigen-binding fragment thereof that binds to mesothelin protein, the antibody, bispecific antibody, or antigen-binding fragment thereof comprising: comprising: (i) a heavy chain variable region comprising: a CDR1, a CDR2, and a CDR3 of the amino acid sequences as disclosed herein; and a light chain variable region comprising: a CDR1, a CDR2, and a CDR3 of the amino acid sequences as disclosed herein.

In some further embodiments of the above aspect, the method comprises administering an antibody, bispecific antibody, or antigen-binding fragment thereof comprising a heavy chain variable region and a light chain variable region comprising an amino acid sequence as described herein.

In some embodiments of the above methods, the cancer comprises a solid tumor cell cancer.

In another aspect, the disclosure provides a method of inhibiting proliferation of a cell that expresses mesothelin (e.g., aberrant expression, over-expression, and the like), the method comprising contacting the cell with an effective amount of an antibody, bispecific antibody, or antigen-binding fragment thereof according to the above aspects and embodiments. In some embodiments of this method, the cell comprises a cancer cell.

In another aspect, the disclosure provides a method of detecting the presence of mesothelin in a biological sample, comprising contacting the biological sample with an effective amount of an antibody, bispecific antibody, or antigen-binding fragment thereof according to the above aspects and embodiments under conditions that allow for formation of a complex between the antibody, bispecific antibody, or antigen-binding fragment thereof and mesothelin present in the sample, and measuring a detectable signal associated with the formation of the complex. In further embodiments of these methods, the antibody, bispecific antibody, or antigen-binding fragment thereof comprises a detectable label.

In any of the above aspects and embodiments relating to methods, some embodiments provide for a subject who is a human, and/or a cell that is a human cell, and/or a biological sample that is from a human.

In another aspect, the disclosure provides an isolated polynucleotide encoding an antibody, bispecific antibody, or antigen-binding fragment thereof according to the above aspects and embodiments.

In another aspect, the disclosure provides an isolated recombinant cell that produces an antibody, bispecific antibody, or antigen-binding fragment thereof according to the above aspects and embodiments.

Other aspects and embodiments of the disclosure will be apparent to one of skill in the art in light of the disclosure and illustrative examples that follow.

### BRIEF DESCRIPTION OF THE DRAWINGS

FIG. 1A-B. Depicts predicted CDR sequences of m912 VH (A) and VL (B). The prediction was obtained by submitting m912 VH and VL sequences to the proABC-2 webserver. The putative CDR residues are highlighted in black bars based on the probability of making intermolecular contacts (pt) with its antigen. Amino acid sequences of the putative CDRs are listed below.

FIG. 2A-F. Depicts consensus sequences presented by sequence logos from m912 affinity maturation experiments. Libraries used in panning and parental CDR sequences are shown on the top and bottom of the figure logos, respectively. The number of sequences used to generate each logo is also shown. Logos represent panning results of individual HCDR3 libraries (A-D), pooled HCDR1 libraries (E), LCDR1 library (F), pooled LCDR3 libraries (G), and the expanded LCDR3 library (H).

FIG. 3A-J. Depicts soluble scFv ELISA analyses of the selected clones from the m912 affinity maturation experiments. The selected clones for constructing combo antibodies are highlighted by bold lines. A-B. ELISA of selected clones from HCDR1 panning. C. Western blot of scFv supernatants of m912 and the selected HCDR1 clones. D-E. ELISA of the selected clones from HCDR3 panning. F. Western blot of scFv supernatants of the selected HCDR3 clones. G. ELISA of the selected clones from LCDR3 panning. H. Western blot of scFv supernatants of m912, HP4-44, LCDR3-2, and the selected LCDR1 clones. I-J. ELISA of selected clones from LCDR1 panning.

FIG. 4A-H. Depicts affinity profiling of MSLN antibodies by Octet using a single concentration of mesothelin. The antibody-captured biosensors were then submerged in wells containing 125 nM mesothelin for 5 min followed by 5 min of dissociation time in the kinetic buffer. m912 was used as a reference in each set of the experiments.

FIG. 5A-C. Depicts non-specific binding of MSLN antibodies to mesothelin negative H929 cells by flow cytometry. A. m912, HP1-A13, and HP4-44 antibodies were used to stain MSLN-positive OVCAR3 cells. Cells stained with Alexa Fluor®647 anti-human IgG Fc secondary antibody alone served as negative controls. Cells were stained by antibodies or antibody supernatants at 5 mg/mL. B. A representative profile of non-specific binding to H929 cells from MSLN-48 in contrast to m912 and HP4-44 staining. C. Ranking non-specific binding of MSLN antibodies to H929 cells using x-means of peaks.

FIG. 6A-C. Depicts affinity measurement of selected MSLN-[L]-scFv^{CD3} bsAbs by Octet. Representative data of the measurements. The Fe-epitope anti-human capture (AHC) biosensors from ForteBio were used for loading the testing bsAbs. A bsAbs were loaded onto probes by dipping into 2.5 μg/ml antibody and followed by a 30 s baseline step. A probe loaded with a control antibody was used as a reference sensor. The antibody-loaded probes were submerged in wells containing a series of concentrations of mesothelin (0 nM, 6.25 nM, 12.5 nM, 25 nM, 50 nM, 100 nM, and 200 nM) for 300 s then followed by 300 s of dissociation time in the kinetics buffer. The dissociation constants (K_{D}) and Kₒₙ/K_{off} are estimated by fitting to the 1:1 binding model. The probes were regenerated for multiple rounds of measurements.

FIG. 7. Depicts affinity measurement of MSLN-39 IgG by Surface Plasmon Resonance (SPR). The measurement was performed by immobilizing the tested antibody and then perfusing with corresponding concentrations of mesothelin. KD and kinetic parameters are listed.

FIG. 8A-D. Depicts cell surface binding of MSLN-[L]-scFv^{CD3} bsAbs. A. Binding to MSLN-positive OVCAR3 cells. B. Binding to MSLN-negative H929 cells. C. OVCAR3 surface binding profiles of MSLN-[L]-scFv^{CD3} bsAbs. D. Jurkat surface binding profiles of MSLN-[L]-scFv^{CD3} bsAbs.

FIG. 9. Depicts sandwich ELISA assay for simultaneous binding of antigens to MSLN-[L]-scFv^{CD3} bsAbs. The assay was performed by coating CD3ε/CD3δ heterodimer on the plate to allow MSLN-[L]-scFv^{CD3} bsAb captured by the CD3 arm. The simultaneous binding of MSLN to the second arm was detected using HRP-conjugated streptavidin which recognizes biotin on the Avi-tag of supplemented MSLN.

FIG. 10A-D. Depicts T cell activation assays of MSLN-[L]-scFv^{CD3} bsAbs in the presence of Capan-2 (A) and OVCAR3 (C), and in the absence of Capan-2 (B) and OVCAR3 (D). Hook effect was observed at high concentrations of MSLN-[L]-scFv^{CD3} bsAbs. For fitting purpose, the data points at high concentrations are masked.

FIG. 11A-B. Depicts KILR cytotoxicity assays of MSLN-[L]-scFv^{CD3} bsAbs in the presence of PBMCs and Capan-2/KILR cells. PBMCs from two donors (A and B) were mixed with Capan2/KILR cells at a ratio of 15:1. Cytotoxicity was detected by monitoring ePL release in the presence of series dilution 5× of MSLN-[L]-scFv^{CD3} bsAbs after 48 hours incubation.

FIG. 12. Depicts luciferase-based cytotoxicity assay of MSLN-[L]-scFv^{CD3} bsAbs in the presence of PBMCs and NCI-N87-Luc cells.

FIG. 13A-B. Depicts influence of soluble MSLN on cytotoxicity effects of MSLN-[L]-scFv^{CD3} bsAbs. The assay was performed using luciferase-based cytotoxicity assay on NCI-N87 cells in the absence and presence of supplemented MSLN. HP4-44 (A) and MSLN-39 (B) of MSLN-[L]-scFv^{CD3} bsAbs were assayed side-by-side.

FIG. 14A-D. Depicts inhibition of tumor growth by MSLN-[L]-scFv^{CD3} bsAbs in NCI-N87/PBMC co-grafting model. A. Schematic depiction of tumor cell inoculation and antibody treatment protocol. B. Tumor growth measured throughout the study for groups of PBS, HPN536 at 0.2 mg/kg, and two samples of HP4-44 and MSLN-39 bsAbs at doses of 0.2 mg/kg, 0.02 mg/kg, and 0.002 mg/kg. Data points represent the mean±standard deviation (SD) of six mice. Statistical analysis was based on two-tailed t-test with * P<0.05 and ***P<0.0001 vs PBS group. C. Images of stripped tumors from individual mice of each groups at the end of the study. D. Body weight measured throughout the study.

FIG. 15. Depicts pharmacokinetic study of MSLN-[L]-scFv^{CD3} bsAbs. Male BALB/c mice were divided into two groups (n=5/group). HP4-44, and MSLN-39 bsAbs were single-intravenously (i.v.) injected into mice with at 5 mg/kg. Mice blood was then collected at the following time points: 0 (predose), 15 min, 6 h, 1, 2, 4, 7, 10, 15, 21 and 28 days. The antibody concentration in blood serum was quantified by ELISA in which human recombinant mesothelin (Acro Biosystems) was immobilized. PK parameters were determined with a noncompartmental analysis model using PKsolver.

### DETAILED DESCRIPTION

The disclosure generally relates to engineered antibodies, and in particular embodiments bispecific antibodies having specific binding to mesothelin, and which have broad utility (i.e., diagnostic and therapeutic applications). The antibodies and bispecific antibodies disclosed herein enhance the potential efficacy of targeting mesothelin in methods of treating diseases (e.g., use in cancer immunotherapy). In some embodiments, the disclosure relates to engineered forms of the m912 antibody that improve its binding affinity to mesothelin. As disclosed herein, engineered forms of m912 have been incorporated into a bispecific format that can efficiently redirect cytotoxic T cells to mesothelin-expressing tumors. The bispecific antibodies in accordance with the example aspects and embodiments disclosed herein can be used for effective treatment against various types of cancer associated with aberrant and/or overexpression of mesothelin. The improved binding affinity exhibited by the antibodies, bispecific antibodies, and fragments thereof that are described herein provide for further antibody formats, such as ADC and immunotoxin, that can be useful in the treatment of cancer.

It is to be understood that this disclosure is not limited to specific proteins, nucleic acids, compositions, methods or process steps that are described below as exemplary and illustrative aspects and embodiments, as variations and modifications fall within the scope of the description.

As used in this specification and the appended claims, the singular form “a”, “an” and “the” include plural referents unless the context clearly dictates otherwise. Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

### A. Antibodies

As used herein, the term “antibody” or its plural, “antibodies”, also known as immunoglobulins, encompass full-length antibody sequences including, for example, monoclonal antibodies (including full-length monoclonal antibodies), polyclonal antibodies, multispecific antibodies formed from at least two different epitope binding fragments, bispecific antibodies, human antibodies, and humanized antibodies. In some embodiments, the disclosure specifically provides a bispecific antibody, or antigen binding fragments thereof. Antibody “fragments” (or “antigen-binding fragments”, “binding fragments”, “epitope-binding fragments”, and the like) as described herein typically refer to any antibody sequence that is less than the full-length antibody sequence, and still exhibits specific binding activity to the target antigen. In example embodiments, antibody fragments typically comprise at least a combination of three CDR sequences of a heavy chain variable domain (HCDR1, HCDR2, HCDR3) and at least three CDR sequences of a light chain variable domain (LCDR1, LCDR2, LCDR3). Some non-limiting examples of antibody fragments include single-chain Fvs (scFv), single chain Fv-Fc (scFv-Fc), single-chain antibodies, single domain antibodies, domain antibodies, Fab fragments, F(ab′)2 fragments, camelised antibodies, antibody fragments that exhibit the desired biological activity (e.g. the antigen binding portion), disulfide-linked Fvs (dsFv), and anti-idiotypic (anti-Id) antibodies, intrabodies, and epitope-binding fragments of any of the above. In some embodiments, the disclosure provides antibodies that include immunoglobulin molecules and immunologically active fragments of immunoglobulin molecules, i.e., molecules that contain at least one antigen-binding site. Antibodies and fragments thereof may also include peptide fusions with antibodies or portions thereof such as a protein fused to an Fc domain.

In accordance with some of the aspects and embodiments described herein, the disclosure provides and relates to bispecific antibodies that have specific binding activity for mesothelin and another targeted sequence.

Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies and fragments thereof may be derived from any mammal, including, but not limited to, humans, monkeys, pigs, horses, rabbits, dogs, cats, mice, etc., or other animals such as birds (e.g. chickens).

In some aspects, the disclosure provides novel binding agents. In some exemplary embodiments, the novel binding proteins are IgGs, scFvs, Fab, monoclonal antibodies (mAbs) or single chain Fv-Fc (scFv-Fc) antibodies. A typical or conventional mAb comprises two heavy chain subunits and two light chain subunits. Each mAb heavy chain contains one variable domain (VH) which contributes to antigen binding and a constant domain (CH) made up of three or four subregions (C_{H}1, C_{H}2, C_{H}3, C_{H}4). The VH comprises three complementarity-determining regions (CDRs), HCDR1, HCDR2, and HCDR3. Each mAb light chain contains one variable domain (VL) and one constant domain (CL). The VL comprises three CDRs, LCDR1, LCDR2, and LCDR3. There are two isotypes of light chain constant domains, kappa (κ) and lambda (λ), found in mammals. Disulfide bonds join each C_{H}1 domain to one CL domain, and join C_{H}2 domains to one another. Five types of heavy chains (α, δ, ε, γ, and μ) are found in different classes of antibodies (IgA, IgD, IgE, IgG, and IgM). mAb heavy chains have hinge regions that confer structural flexibility and mobility.

The “Fc” region encompasses domains from the constant region of the heavy chain of an immunoglobulin, including a fragment, analog, variant, mutant, or derivative thereof. Suitable immunoglobulins include IgG1, IgG2, IgG3, IgG4, and other classes such as IgA, IgD, IgE and IgM. The Fc region may be a native sequence Fc region or an altered Fc region. The Fc region of an immunoglobulin generally comprises two constant domains, a C_{H}2 domain and a C_{H}3 domain. The “Fv” region encompasses the VH and VL domains of an immunoglobulin. As used herein, “scFv-Fc” antibodies refer to a fusion protein of a single VH and a single VL domain, connected with a hinge region, and the C_{H}2 domain and the C_{H}3 domain of a single CH domain.

As discussed herein, the antibodies, bispecific antibodies, and antigen-binding fragments thereof disclosed herein comprise binding domains that bind to an epitope of mesothelin. “Binding domain” or “binding sequence” may be interchangeably used with an antibody fragment, bispecific antibody fragment, or “antigen-binding fragment”, and as used herein refers to a portion of an specific binding sequence that is adequate and sufficient to bind to or to interact with a target structure, antigen, or epitope. In some aspects, the antibodies bind to a region of mesothelin that comprises an antigenic sequence or fragment. In other aspects, the antibodies bind to a domain of the mesothelin protein (e.g., region I, region II, or region III).

In some embodiments the antibodies, bispecific antibodies, and antigen-binding fragments thereof in accordance with the disclosure bind to mesothelin, and can inhibit its binding interactions with other molecules and/or one or more biological functions. In some example embodiments the antibodies, bispecific antibodies, and antigen-binding fragments thereof can inhibit or antagonize one or more mesothelin-associated biological pathways. In accordance with such example embodiments of the disclosure, the antibodies, bispecific antibodies, or fragments thereof can prevent, inhibit, or reduce binding of and/or activity of mesothelin, and may provide neutralizing activity against mesothelin as well as its downstream signaling partners and pathways.

In some embodiments, the antibodies and bispecific antibodies disclosed herein can be characterized by one or more of the following structural and/or functional properties:
a. an amino acid sequence comprising a VH domain and a VL domain, wherein the VH domain comprises an HCDR1, HCDR2, and HCDR3 identified by the SEQ ID NOs disclosed herein; and wherein the VL domain comprises a LCDR1, a LCDR2, and an LCDR3 identified by the SEQ ID NOs disclosed herein;
b. an amino acid sequence comprising a VH domain having the amino acid sequence of the SEQ ID NOs disclosed herein; and/or a VL domain having the amino acid sequence of the SEQ ID NOs disclosed herein;
c. an amino acid sequence having an HCDR1 comprising SEQ ID NOs: 85, 86, 97, 49, 53, 57, 61, 65, 69, 73, 77, or 81; an HCDR2 comprising SEQ ID NOs: 50, 54, 58, 62, 66, 70, 74, 78, 82, 87, 88, or 98; an HCDR3 comprising SEQ ID NOs: 51, 55, 59, 63, 67, 71, 75, 79, 83, 89, 90, 91, 99, 100, or 101; and/or an LCDR1 comprising SEQ ID NOs: 93; an LCDR 2 comprising SEQ ID NOs: 94; an LCDR2 comprising SEQ ID NOs: 95 or 96.
d. binding specificity for mesothelin;
e. binding specificity for mesothelin and another targeted sequence;
f. regarding mesothelin, a dissociation constant (K_{D}) in the range of 1 pM-500 nM, 10 pM-500 nM, 0.1 nM-500 nM, 1-450 nM, 1-400 nM, 1-300 nM, 1-200 nM, 1-100 nM, 1-50 nM, 1-25 nM, or 1-10 nM;
g. regarding mesothelin, an IC50 value in the range of 0.01-250 nM, 0.01-200 nM, 0.01-150 nM, 0.01-100 nM, 0.01-50 nM, 0.01-25 nM, 0.01-20 nM, or 0.01-10 nM;
h. the ability to inhibit, reduce, prevent, or disrupt mesothelin binding interactions, as may be measured by an assay including, for example, filter binding assay, fluorescence spectroscopy, ELISA, isothermal titration calorimetry, and surface plasma resonance.

In some aspects, an antibody, bispecific antibody, or antigen-binding fragment thereof that is capable of inhibiting, reducing, preventing, or disrupting the binding activity or biological activity of mesothelin, and/or capable of neutralizing the activity of mesothelin, binds to an epitope present in the mesothelin protein.

In some aspects, an antibody, bispecific antibody, that binds mesothelin, or an antigen-binding fragment thereof, comprises a VH domain having the amino acid sequence as described herein.

In some aspects, an antibody, bispecific antibody, that binds mesothelin, or an antigen-binding fragment thereof, comprises a VL domain having the amino acid sequence as described herein

In certain embodiments of these aspects, an antibody, or an antigen-binding fragment thereof, comprises a VH domain and VL domain having the combinations of heavy chain and light chains, or heavy and light chain CDR sequences that are identified in Tables and identified by SEQ ID NOs.

In exemplary embodiments, the antibodies, bispecific antibodies, or fragments thereof that are disclosed herein exhibit binding specificity for the mesothelin protein, and can inhibit, reduce, prevent, or disrupt mesothelin binding interactions. In further embodiments, the antibodies, bispecific antibodies, or fragments thereof bind to mesothelin and another protein target.

As one of ordinary skill can appreciate, the sequences disclosed herein can be modified to some degree without compromising the ability of the antibody or fragment thereof to interact with mesothelin. In some aspects, antibody or bispecific antibody sequence variants retain the ability to interact with mesothelin such that the binding interaction between mesothelin and any one or more of its binding partners is disrupted, prevented, reduced, or inhibited.

As used herein, sequence “variants” refer to an antibody amino acid sequence comprising at least one amino acid insertion, deletion, and/or substitution, wherein the resulting antibody maintains one or more of its functional characteristics as described herein. In embodiments, a sequence variant maintains the ability to specifically bind mesothelin. An amino acid insertion variant is characterized by the insertion of one or more amino acids between two existing amino acids. An amino acid deletion variant is characterized by the deletion of one or more amino acids from the antibody sequence. An amino acid substitution is characterized by at least one amino acid in the sequence being replaced by another amino acid. In embodiments relating to substitutions, the amino acid substitution(s) may be a conservative substitution, (i.e. an amino acid from one family of amino acids (acidic, basic, non-polar, and uncharged, based on side chain characteristics, including size) is substituted with an amino acid from the same family).

In some aspects, the sequence identity between the variant antibody sequence and the antibody sequences disclosed herein will be at least 85%, 86%, 87%, 88%, 89%, 90%, 91%, 92%, 93%, 94%, 95%, 96%, 97%, 98%, or 99%. “Sequence identity” refers to the percentage of amino acid residues that are identical to the sequences being compared.

### B. Labels, Conjugates, and Moieties

The binding proteins, antibodies, bispecific antibodies, or antigen binding fragments thereof disclosed herein can be conjugated to a therapeutic agent, solid support, affinity agent, or a detectable agent. For example, the amino acid sequences disclosed herein may be conjugated to labels for the purposes of diagnostics and other assays wherein the amino acid sequence (i.e., antibody and/or its associated targets(s)) may be detected. Labels include, without limitation, chromophores, fluorophores, fluorescent proteins, phosphorescent dyes, tandem dyes, particles (micro- and nano-particles of various materials (polymer, magnetic, etc.), haptens, enzymes, peptides, and radioisotopes, among others, and combinations thereof.

In certain aspects, the antibodies or bispecific antibodies are conjugated to a fluorophore. The choice of the fluorophore attached to the antibody will determine the absorption and fluorescence emission properties of the conjugated antibody. Physical properties of a fluorophore label that can be used for an antibody and antibody-bound ligands include, but are not limited to, spectral characteristics (absorption, emission and stokes shift), fluorescence intensity, lifetime, polarization and photo-bleaching rate, or combination thereof. All of these physical properties can be used to distinguish one fluorophore from another, and thereby allow for multiplexed analysis. Other desirable properties of the fluorescent label may include cell permeability and low toxicity, for example if labeling of the antibody is to be performed in a cell or an organism (e.g., a living animal).

In certain aspects, the conjugated label may comprise an enzyme. Enzymes are desirable labels in some embodiments because amplification of the detectable signal can be obtained and result in increased assay sensitivity. The enzyme itself does not produce a detectable response but functions to break down a substrate when it is contacted by an appropriate substrate such that the converted substrate produces a fluorescent, colorimetric or luminescent signal. Enzymes may amplify the detectable signal because one enzyme on a labeling reagent can result in multiple substrates being converted to a detectable signal. The enzyme substrate is selected to yield the preferred measurable product, e.g. colorimetric, fluorescent or chemiluminescence. Such substrates are extensively used in the art and are well known by one skilled in the art and include for example, oxidoreductases such as horseradish peroxidase and a substrate such as 3,3′-diaminobenzidine (DAB); phosphatase enzymes such as an acid phosphatase, alkaline and a substrate such as 5-bromo-6-chloro-3-indolyl phosphate (BCIP); glycosidases, such as beta-galactosidase, beta-glucuronidase or beta-glucosidase and a substrate such as 5-bromo-4-chloro-3-indolyl beta-D-galactopyranoside (X-gal); additional enzymes include hydrolases such as cholinesterases and peptidases, oxidases such as glucose oxidase and cytochrome oxidases, and reductases for which suitable substrates are known.

Enzymes and their appropriate substrates that produce chemiluminescence are suitable for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In another aspect, haptens, such as biotin, are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

In certain aspects, fluorescent proteins may be conjugated to the antibody as a label. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra.

In certain aspects, the label is a radioactive isotope. Examples of suitable radioactive materials include, but are not limited to, iodine (¹²¹I, ¹²³, ¹²⁵I, ¹³¹I), carbon (¹⁴C), sulfur (³⁵S), tritium (³H), indium (¹¹¹In, ¹¹²In, ¹¹³mIn, ¹¹⁵mIn,), technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³⁵Xe), fluorine (¹⁸F), ¹⁵³SM, ¹⁷⁷Lu, ¹⁵⁹Gd, ¹⁴⁹Pm, ¹⁴La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh and ⁹⁷Ru.

In some aspects, drugs may be conjugated to the antibody or bispecific antibodies. For example, bispecific antibody may be conjugated to a therapeutic moiety or agent, such as an anti-proliferative, anti-cancer, cytotoxin, or antiviral drug. In some embodiments, the antibody may bind to a target antigen, and the drug (e.g., cancer drug) acts to inhibit, inactivate, or kill a cancer cell. Accordingly, any drug known in the art may be conjugated to an antibody, or fragment thereof, in accordance with the disclosure. In certain embodiments, drugs and other molecules may be conjugated to an antibody or bispecific antibody via site-specific conjugation.

### C. Polynucleotides Encoding Antibodies & Recombinant Cells Producing Antibodies

The disclosure provides methods for producing antibodies, bispecific antibodies, and antibody fragments. In certain aspects, the disclosure provides for recombinant methods of generating the antibodies and/or fragments thereof. In some embodiments, recombinant nucleic acids encoding for the antibody or bispecific antibody, or fragment thereof may be operably linked to one or more regulatory nucleotide sequences in an expression construct. In some embodiments, the nucleic acid sequences encoding the antibody light and heavy chains can be cloned in the same expression vector in any orientation (e.g., light chain in front of the heavy chain or vice versa) or can be cloned in two different vectors. If expression is carried out using one vector, the two coding genes can have their own genetic elements (e.g., promoter, RBS, leader, stop, polyA, etc.) or they can be cloned with one single set of genetic elements, but connected with a cistron element. Regulatory nucleotide sequences may be appropriate for a host cell used for expression. Numerous types of appropriate expression vectors and suitable regulatory sequences are known in the art for a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Any known constitutive or inducible promoters are contemplated for use with the aspects and embodiments included in the disclosure. The promoters may be either naturally occurring promoters, or hybrid promoters that combine elements of more than one promoter. An expression construct may be present in a cell on an episome, such as a plasmid, or the expression construct may be inserted in a chromosome.

In certain aspects, an expression vector contains a selectable marker gene to allow the selection of transformed host cells. Selectable marker genes are known and may vary with the host cell used. In certain aspects, the disclosure relates to an expression vector comprising a nucleotide sequence encoding a polypeptide that is operably linked to at least one regulatory sequence. Regulatory sequences are generally known and may be selected to direct expression of the encoded polypeptide. Accordingly, the term regulatory sequence includes promoters, enhancers, and other expression control elements. Exemplary, non-limiting regulatory sequences are described in Goeddel; Gene Expression Technology: Methods in Enzymology, Academic Press, San Diego, CA (1990). It should be understood that the design of the expression vector may depend on such factors as the choice of the host cell to be transformed and/or the type of protein (e.g., antibody or fragment thereof) to be expressed. Moreover, the vector's copy number, the ability to control that copy number and the expression of any other protein encoded by the vector, such as antibiotic markers, may be considered.

In some embodiments relating to methods of producing an antibody, a host cell may be transfected with one or more expression vector(s) encoding an antibody or fragment thereof (e.g., a single vector encoding the heavy and the light chain or two vectors, one encoding the heavy chain and one encoding the light chain) and can be cultured under appropriate conditions to allow expression of the polypeptide to occur. The antibody or fragment thereof may be secreted and isolated from cells and/or cell culture media containing the antibody or fragment thereof. Alternatively, the antibody may be retained in the cytoplasm or in a membrane fraction and the harvested cells, which may be lysed subsequently purified and isolated. A cell culture includes host cells, media and other byproducts. Any suitable media for cell culture may be used in methods of production. Antibodies and antibody fragments can be isolated from cell culture medium, host cells, or both using common techniques for purifying proteins, and antibodies in particular, including, for example, ion-exchange chromatography, gel filtration chromatography, ultrafiltration, electrophoresis, and immunoaffinity purification. In certain aspects, the antibody may be produced as a fusion protein containing a domain (e.g., a His-tag) which may facilitate its purification.

A recombinant nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. In certain aspects, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various nucleic acid fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another aspect, the fusion gene can be synthesized by conventional techniques including automated DNA synthesizers. Alternatively, PCR amplification of gene fragments can be carried out using anchor primers which give rise to complementary overhangs between two consecutive nucleic acid fragments which can subsequently be annealed to generate a chimeric gene sequence (see, for example, Current Protocols in Molecular Biology, eds. Ausubel et al., John Wiley & Sons: 1992).

In some aspects, an expression vector expressing any of the nucleic acids described above may be used to express an antibody or bispecific antibody in a host cell. For example, an antibody may be expressed in bacterial cells such as E. coli, insect cells (e.g., using a baculovirus expression system), yeast, or mammalian cells. Other suitable host cells are known to those skilled in the art.

Once the expression vector is transferred to a host cell by conventional techniques, the transfected cells are then cultured by conventional techniques to produce an antibody or bispecific antibody. Thus, the disclosure includes host cells containing a polynucleotide encoding an antibody or antigen-binding fragments thereof, operably linked to a heterologous promoter. In certain aspects, both the heavy chain and the light chain may be co-expressed (from the same or different vectors) in the host cell for expression of the entire antibody. In certain aspects, both the heavy and light chains of the antibody or bispecific are expressed from a single promoter. In certain aspects, the heavy and light chains of the antibody or bispecific antibody are expressed from multiple promoters. In certain aspects, the heavy and light chains of the antibody are encoded on a single vector. In certain aspects, the heavy and light chains of the antibody or bispecific antibody are encoded on multiple vectors.

Mammalian cell lines available as hosts for expression of recombinant antibodies are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Different host cells have characteristic and specific mechanisms for the post-translational processing and modification of proteins and gene products. Appropriate cell lines or host systems can be chosen to ensure the correct modification and processing of the antibody or portion thereof expressed. To this end, eukaryotic host cells that possess the cellular machinery for proper processing of the primary transcript, glycosylation, and phosphorylation of the gene product may be used. Such mammalian host cells include but are not limited to CHO, HEK293, VERO, BHK, Hela, COS, MDCK, 293, 3T3, W138, BT483, Hs578T, HTB2, BT2O and T47D, NS0 (a murine myeloma cell line that does not endogenously produce any functional immunoglobulin chains), SP20, CRL7030 and HsS78Bst cells.

In certain aspects, antibodies, bispecific antibodies, and antibody fragments of the disclosure are stably expressed in a cell line. Stable expression can be used for long-term, high-yield production of recombinant proteins. For example, cell lines that stably express the antibody or bispecific antibody molecule may be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells may be allowed to grow for 1-2 days in an enriched media, and then are switched to a selective media. The selectable marker in the recombinant plasmid confers resistance to the selection and allows cells that stably integrated the plasmid into their chromosomes to grow and form foci, which in turn can be cloned and expanded into cell lines. Methods for producing stable cell lines with a high yield are well known in the art and reagents are generally available commercially.

In certain aspects, antibodies, bispecific antibodies, and antibody fragments of the disclosure are transiently expressed in a cell line. Transient transfection is a process in which the nucleic acid introduced into a cell does not integrate into the genome or chromosomal DNA of that cell, but is maintained as an extrachromosomal element, e.g. as an episome, in the cell. Transcription processes of the nucleic acid of the episome are not affected and a protein encoded by the nucleic acid of the episome is produced.

The cell line, either stable or transiently transfected, is maintained in cell culture medium and conditions well known in the art resulting in the expression and production of monoclonal antibodies. In certain aspects, the mammalian cell culture media is based on commercially available media formulations, including, for example, DMEM or Ham's F12. In other aspects, the cell culture media is modified to support increases in both cell growth and biologic protein expression. As used herein, the terms “cell culture medium,” “culture medium,” and “medium formulation” refer to a nutritive solution for the maintenance, growth, propagation, or expansion of cells in an artificial in vitro environment outside of a multicellular organism or tissue. Cell culture medium may be optimized for a specific cell culture use, including, for example, cell culture growth medium that is formulated to promote cellular growth, or cell culture production medium which is formulated to promote recombinant protein production. The terms nutrient, ingredient, and component are used interchangeably herein to refer to the constituents that make up a cell culture medium.

Once an antibody or bispecific antibody has been produced, it may be purified by any method known in the art for purification of an immunoglobulin molecule or other multimeric molecules, for example, by chromatography (e.g., ion exchange, affinity, particularly by affinity for the specific antigens Protein A or Protein G, and sizing column chromatography), centrifugation, differential solubility, or by any other standard technique for the purification of proteins.

When using recombinant techniques, the antibody, bispecific antibody, and antibody fragment can be produced intracellularly, in the periplasmic space, or directly secreted into the medium. If the molecule is produced intracellularly, as a first step, the particulate debris, either host cells or lysed fragments, is removed, for example, by centrifugation or ultrafiltration. Carter et al., Bio/Technology, 10:163-167 (1992) describe a procedure for isolating antibodies, which are secreted into the periplasmic space of E. coli. Where the molecule is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The composition prepared from the cells can be purified using, for example, hydroxylapatite chromatography, hydrophobic interaction chromatography, ion exchange chromatography, gel electrophoresis, dialysis, and/or affinity chromatography either alone or in combination with other purification steps. The suitability of protein A as an affinity ligand depends on the species and isotype of any immunoglobulin Fc domain, if present, in the molecule and will be understood by one of skill in the art. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available depending on the molecule to be recovered.

Regardless of how an antibody, bispecific antibody, or and antibody fragments is purified, to confirm functional binding activity, binding assays may be performed (before and/or after purification). For example, ELISA assays, including dual ELISA assays may be used. In some aspects, a first antigen is coated on a well, and binding to this antigen immobilizes the antibody. A tagged second antigen is added to the well, and detected. Only antibodies that are both immobilized via binding to the first antigen and bound to the second antigen will be detected, In some aspects, the disclosure provides for recombinant cell lines that may be deposited and maintained with an international depository institution that is authorized under the provisions of the Budapest Treaty (i.e., an International Depositary Authority, IDA).

### D. Pharmaceutical Formulations

In certain aspects, the disclosure provides pharmaceutical compositions. Such pharmaceutical compositions may also be compositions comprising an antibody, bispecific antibody, and/or an antibody fragment as disclosed herein and a pharmaceutically acceptable excipient. In certain aspects, the pharmaceutical compositions of the disclosure are used as a medicament (i.e., in methods of treating or preventing a disease or condition (e.g., cancer or a clinically related symptom thereof), in a subject in need of treatment or preventative treatment). In some embodiments, pharmaceutical compositions may be compositions comprising a nucleic acid molecule that encodes an antibody as disclosed herein.

In certain aspects, an antibody, bispecific antibody, (or nucleic acid molecules encoding an antibody) may be formulated with a pharmaceutically acceptable carrier, excipient or stabilizer, as pharmaceutical compositions. In certain aspects, such pharmaceutical compositions are suitable for administration to a human, or a non-human mammal or animal, via any one or more route of administration using methods known in the art. The route and/or mode of administration will vary depending upon the desired results. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances, which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium and the like. These and additional known pharmaceutical carriers, excipients and/or additives suitable for use in the formulations described herein are known in the art, e.g., as listed in “Remington: The Science & Practice of Pharmacy”, 21^{st} ed., Lippincott Williams & Wilkins, (2005), and in the “Physician's Desk Reference”, 60^{th} ed., Medical Economics, Montvale, N.J. (2005). Pharmaceutically acceptable carriers can be selected that are suitable for the mode of administration, solubility and/or stability desired or required.

The formulations described herein comprise active agents (i.e., one or more antibody, bispecific antibody, or fragments thereof as disclosed herein) in a concentration resulting in a w/v appropriate for a desired dose. In certain aspects, the active agent is present in a formulation at a concentration of about 1 mg/ml to about 200 mg/ml, about 1 mg/ml to about 100 mg/ml, about 1 mg/ml to about 50 mg/ml, or about 1 mg/ml to about 25 mg/ml. In certain aspects, the concentration of the active agent in a formulation may vary from about 0.1% to about 75% by total weight. In certain aspects, the concentration of the active agent is in the range of 0.003 to 1.0 molar.

When used for in vivo administration, the formulations should be sterile. Formulations may be sterilized by various sterilization methods, including sterile filtration, radiation, etc. In one aspect, the formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005).

Thus, in embodiments, therapeutic compositions are encompassed by the pharmaceutical formulations described herein, and may formulated for particular routes of administration, such as oral, nasal, pulmonary, topical (including buccal and sublingual), rectal, vaginal and/or parenteral administration. The phrases “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The formulations may be in unit dosage form and may be prepared by any known method. Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. Suitable dosages may range from about 0.0001 to about 100 mg/kg of body weight or greater, for example about 0.1, 1, 10, or 50 mg/kg of body weight, with about 1 to about 10 mg/kg of body weight being suitable.

In some embodiments of the disclosure, the formulations may be suitable for diagnostic and research use. The concentration of active agent in such formulations, as well as the presence or absence of excipients and/or pyrogens, can be modified or selected based on the particular application and intended use.

### E. Methods

The antibodies, bispecific antibodies, and fragments thereof, as described herein, can be used in methods that prevent, inhibit, or reduce the activity and/or expression of mesothelin in a subject. In this aspect, the antibodies, bispecific antibodies, and fragments thereof disclosed herein can provide a neutralizing effect against mesothelin. In some aspects, the antibodies or fragments of the disclosure can be used to treat a mesothelin-related condition, disease, or disorder in a subject in need of treatment. In some aspects, the antibodies, bispecific antibodies, and fragments disclosed herein can be used to prevent a mesothelin-associated condition, disease, or disorder in a subject, who may be at risk of developing a condition, disease, or disorder. In some aspects, the antibodies or bispecific antibodies can be used to treat cancer associated with mesothelin activity and/or expression, in a subject in need of treatment.

In some aspects, the disclosure relates to methods of treating, preventing, diagnosing, or monitoring, a condition, disease, or disorder characterized by mesothelin, and in some embodiments the disease is a cancer. In such aspects, the antibodies, bispecific antibodies, compositions and methods described herein can be used to treat a subject with a mesothelin associated condition, disease, or disorder, e.g., a cancer characterized by expression and/or activity of mesothelin.

In embodiments, the disclosure provides a method of treatment comprising an antibody, bispecific antibody, or antigen-binding fragment thereof in accordance with the disclosure can provide for effective treatment or “disease control” (DC). Disease control can be a complete response (CR), partial response (PR), or stable disease (SD).

A “complete response” (CR) refers to the disappearance of all tumors or lesions, whether measurable or not, and formation of no new tumor or lesions. Confirmation can be obtained using a repeat, consecutive assessment (e.g., no less than a defined number of weeks (e.g., 4) from the date of first documentation). New, non-measurable tumors or lesions preclude CR.

A “partial response” (PR) refers to a decrease in tumor burden >50% relative to baseline. Confirmation can be obtained using a consecutive repeat assessment (e.g., at least a defined number of weeks (e.g., 4) from the date of first documentation).

“Progressive disease” (PD) refers to an increase in tumor burden >25% relative to the minimum recorded (nadir). Confirmation can be obtained by a consecutive repeat assessment (e.g., at least a defined number of weeks (e.g., 4) from the date of first documentation). New, non-measurable lesions do not define PD.

“Stable disease” (SD) refers to not meeting the criteria for CR, PR, or PD. SD indicates a decrease in tumor burden of 50% relative to baseline cannot be established and a 25% increase compared to nadir cannot be established.

By “disease” is meant any condition or disorder that damages or interferes with the normal function of a cell, tissue, or organ. In the aspects and embodiments disclosed herein, the disease is typically a cancer including, for example, a solid tumor cancer. In some embodiments, the cancer may include ovarian cancer, pancreatic cancer, gastric, esophageal, gastroesophageal, or mesothelioma.

In some embodiments of the various aspects, the disclosure relates to the determination of the presence, level of expression, and/or activity of mesothelin in samples obtained from a subject who may have a cancer, which can be used to identify patients who are likely to respond to treatment with an antibody in accordance with the disclosure. In some embodiments, the determination may comprise detecting mRNAs encoding mesothelin, and can be used to identify patients who may be responsive to treatment with a mesothelin antibody or bispecific antibody. In some embodiments, the methods disclosed herein comprise detecting the amount and/or activity of mesothelin to determine the responsiveness of a cancer to mesothelin-based immunotherapy. In some further embodiments, the methods disclosed herein comprise detecting the amount and/or activity of mesothelin protein by contacting a sample (i.e., patient sample) with an antibody, bispecific antibody, or antigen-binding fragment thereof in accordance with the disclosure, and detecting the presence (e.g., amount and/or activity of mesothelin) in the sample. In such embodiments, the methods can be used to assess or determine the responsiveness of a cancer to immunotherapy comprising a mesothelin antibody or bispecific. The methods disclosed herein can comprise analysis of blood samples, biopsies, or immunohistochemical techniques applied to individual tissue and/or tumor samples. In some embodiments of this aspect, a second biomarker associated with cancer may be detected at the same time or separate from the detection of mesothelin.

In one aspect, the disclosure provides a method of treating cancer in a patient comprising administering an antibody, bispecific antibody, or an antigen-binding fragment thereof as disclosed herein to the patient, wherein the patient is identified for treatment by detecting mesothelin in a sample obtained from the patient.

As used herein “treat”, “treating”, or “treatment” refers to administering an antibody or antigen binding fragment thereof, or compositions as described herein to a subject in order to eliminate or reduce the clinical signs of a condition, disease, or disorder; arrest, inhibit, or slow the progression of a condition, disease, or disorder in a subject; and/or decrease the number, frequency, or severity of the clinical symptoms and/or recurrence of a condition, disease, or disorder in a subject who currently has or who previously had a condition, disease, or disorder (e.g., a cancer and/or a recurrence of cancer). In particular, the term “treatment of a disease” (e.g., disease associated with cell proliferation, cancer, etc.) includes stopping, ameliorating, reducing, shortening the duration, slowing down, or inhibiting progression or worsening, or delaying the onset of a disease or the symptoms thereof in a subject who has the disease.

In some embodiments, the disclosure provides methods for preventing disease (i.e., preventing a condition, disease, or disorder, preventing the onset of a condition, disease, or disorder or the clinical symptoms of disease) in a subject. In some further embodiments of the methods relating to preventing disease, the subject may be at risk of one or more mesothelin-related conditions, diseases, or disorders. By “being at risk” or having an “increased risk” a subject is identified as having a higher than normal chance of developing a condition, disease, or disorder and/or clinical symptoms associated therewith, compared to the general population. In some embodiments, a subject who has had, or who currently has, a condition, disease, or disorder, or who is of a certain age is a subject who has an increased risk for developing a condition, disease, or disorder. In some aspects, a subject exhibiting increased mesothelin expression or activity can also be considered as having an increased risk for developing a condition, disease, or disorder. In some embodiments the subject may also be immunocompromised. As used herein “immunocompromised” refers to a subject having a weakened immune system or a reduced ability to fight infections or other diseases, due to a genetic disorder or disease, an infection, an environmental disorder or disease, or other environmental factors.

The term “immunotherapy” relates to a treatment involving a specific immune reaction. In the context of the present invention, terms such as “protect”, “prevent”, “prophylactic”, “preventive”, or “protective” relate to the prevention of the occurrence and/or the propagation of a condition, disease, or disorder and clinical symptoms in an individual and, in some embodiments, to minimizing the chance that a subject will develop further clinical symptoms or disease progression. For example, a person at risk for a condition, disease, or disorder associated with mesothelin, as described above, would be a candidate for preventative therapy to prevent such a condition, disease, or disorder or associated clinical symptom(s).

A prophylactic administration of an immunotherapy, for example, a prophylactic administration of an antibody, bispecific antibody, or a composition comprising the antibody or fragment thereof as disclosed herein, can in certain embodiments protect the recipient from a condition, disease, or disorder associated with mesothelin, or reduce the risk of the recipient from developing such a condition, disease, or disorder. In some alternative embodiments, a prophylactic administration may reduce chances of developing increased severity of a condition, disease, or disorder associated with mesothelin in the recipient.

A therapeutic administration of an immunotherapy, for example, a therapeutic administration of an antibody, bispecific antibody or a composition comprising the antibody, bispecific antibody, or fragment thereof as disclosed herein, may lead to the inhibition of the progress of the condition, disease, or disorder and/or clinical symptoms associated with the condition, disease, or disorder. Such methods may include embodiments that comprise the reduction or inhibition of the expression of mesothelin, the activity of mesothelin, and/or the total amount of cells associated with mesothelin expression which preferably leads to elimination of the condition, disease, or disorder and the associated clinical symptoms.

In certain aspects, the disclosure provides methods of treatment comprising a mesothelin antibody, bispecific antibody, or an antigen-binding fragment thereof administered in combination or in conjunction with additional cancer therapies.

The terms “subject”, “individual” or “patient” are interchangeable, and relate to vertebrates, preferably mammals. For example, mammals in the context of the disclosure are humans, non-human primates, domesticated animals such as dogs, cats, sheep, cattle, goats, pigs, horses etc., laboratory animals such as mice, rats, rabbits, guinea pigs, etc., as well as animals in captivity such as animals in zoos. The term “animal” as used herein also includes humans. The term “subject” may also include a patient, i.e., an animal, an in particular embodiments a human having a disease associated with mesothelin. Subjects may also include a patient who may be at risk of developing a condition, disease, or disorder associated with mesothelin. In particular embodiments the subject, individual, or patient is a human.

The methods comprising the antibodies or binding fragments thereof as described herein, and compositions comprising the same, may be administered via any conventional route, including by injection or infusion, oral, buccal, sublingual, transdermal, intraocularly, intranasally, by aerosol, by implant or depot, or intrarectally. In some embodiments, the administration may be carried out, for example, by injection such as, intravenously, intraperitonealy, intramuscularly, subcutaneously, or transdermally.

The antibodies, bispecific antibodies, or fragments thereof, and the compositions comprising them are administered in effective amounts. An “effective amount” includes an amount that achieves a desired reaction or a desired effect and may be in the form of a single dose or as multiple doses. In the case of treatment of a particular disease or of a particular clinical condition, the desired reaction relates to inhibition of the course of the disease or symptom. Such inhibition can include slowing down the progression of the disease/clinical symptoms and, in some embodiments, interrupting or reversing the progression of the disease/clinical symptoms. The treatment of a disease or of a condition may also be delay of the onset or a prevention of the onset of said disease or said condition in a subject who has active disease such as, for example, a cancer.

An effective amount of a composition of the invention will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses of the compositions of the invention administered may depend on various combinations of such parameters. In embodiments in which an initial amount administered to a patient is insufficient, further administration with higher amounts, more frequent doses, or a different/more localized route of administration may be used.

In accordance with the above aspects, in some embodiments the disclosure relates to methods of monitoring a condition, disease, or disorder associated with mesothelin in a subject, wherein the methods comprise the detection and/or the determination or the monitoring of the quantity of (i) a mesothelin nucleic acid, (ii) a mesothelin antigen or a part thereof, (iii) an antibody against mesothelin or a part thereof, and/or (iv) a cell comprising mesothelin, in a biologic sample isolated from a patient. In some further embodiments, the condition, disease, or disorder associated with mesothelin comprises a cancer.

In accordance with the above aspects, in some embodiments the disclosure relates to a method of diagnosing a condition, disease, or disorder characterized by mesothelin expression. In some embodiments, the method comprises the detection, and/or the determination of the quantity, of (i) a mesothelin nucleic acid, (ii) a mesothelin antigen or a part thereof, (iii) an antibody against mesothelin or a part thereof, and/or (iv) a cell comprising mesothelin, in a biologic sample isolated from a patient. In some further embodiments, the condition, disease, or disorder associated with mesothelin expression comprises a cancer.

In some embodiments of the above aspects, detection comprises (i) contacting the biological sample with an antibody, bispecific antibody, or antigen-binding fragment thereof as disclosed herein, which binds specifically to mesothelin or a part thereof, an antibody against mesothelin or a part thereof, and/or a cell comprising surface expression of at least a portion of mesothelin antigen, and (ii) detecting the formation of a complex between the antibody or antigen-binding fragment thereof and mesothelin or the part thereof, the antibody against mesothelin or a part thereof, and/or a cell comprising a mesothelin antigen. In some embodiments, the biological sample isolated from the patient is compared to a reference sample, e.g., a comparable biological sample obtained from a healthy subject having normal levels of G mesothelin expression.

In a further aspect, the disclosure relates to a method for determining regression, course, or onset of a disease characterized by mesothelin. In some embodiments the disease may be identified according to various methods provided by the disclosure. In some embodiments, the method comprises monitoring a sample from a patient who has said disease or is suspected of having said disease, for one or more parameters selected from the group consisting of (i) the amount of nucleic acid associated with mesothelin, (ii) the amount of expressed mesothelin antigen or a part thereof, (iii) the amount of an antibody against mesothelin antigen or a part thereof, and/or (iv) the amount of a cell associated with mesothelin expression. In some, embodiments the method comprises determining the parameter(s) in a first sample at a first point in time and in a further sample at a second point in time and in which the course of the disease is determined by comparing the two samples.

The term “sample” can include any sample that is useful or usable in accordance with the methods described herein. In some embodiments a sample can be a biological sample, such as a sample from a tissue, which may include, for example, bodily fluids, and/or cellular samples that may be obtained by any conventional method (e.g., by tumor/tissue biopsy, taking blood, bronchial aspirate, sputum, urine, feces or other body fluids). In some embodiments, the term “sample” may include processed samples such as fractions or isolates of biological samples, e.g. nucleic acid and peptide/protein isolates.

According to some embodiments, detection of a mesothelin antigen, or associated cell expressing a mesothelin antigen or of a fragment thereof, or determining or monitoring the amount of a mesothelin antigen or cell associated with at least a portion of a mesothelin antigen may be carried out using one or more antibodies, bispecific antibodies, constructs, or fragments as described herein. The methods comprising detection that incorporate an antibody, bispecific antibody, or fragment thereof as disclosed herein may comprise a detectable label. In some embodiments, the detectable label or marker is a fluorescent, colorimetric, radioactive, or an enzymatic marker, or other markers as disclosed herein or otherwise known in the art.

As discussed above, the methods provided herein can decrease, slow, or halt tumor growth and, in some aspects the reduction or slowing may be statistically significant. A reduction in tumor growth can be measured by comparison to the growth of patient's tumor at baseline, against an expected tumor growth, against an expected tumor growth based on a large patient population, or against the tumor growth of a control population.

In certain aspects, administration of a mesothelin antibody, bispecific antibody, or an antigen-binding fragment thereof can increase overall survival (OS). In other aspects, administration of a mesothelin antibody, bispecific antibody or an antigen-binding fragment thereof can provide stable disease (SD).

### F. Kits

Another aspect of the present disclosure is a kit. In one aspect, a kit comprises any of the sequences, compounds and pharmaceutical formulations or compositions of a nucleic acid, polypeptide, expression vector, or host cell as generally described above, and instructions or a label directing appropriate use or administration. Optionally, a kit may also include one or more containers reagents, reactants, and/or a syringe or other device to facilitate delivery or use. The disclosure contemplates that all or any subset of the components for conducting research assays, diagnostic assays and/or for administering therapeutically effective amounts may be enclosed in the kit. Similarly, the kit may include instructions for making an amino acid sequence/polypeptide by, for example culturing a host cell that expresses a nucleic acid that encodes an antibody (or construct or antigen binding fragment thereof) of the disclosure under suitable conditions. By way of additional example, a kit for therapeutic administration of an antibody of the disclosure may comprise a solution containing a pharmaceutical formulation of the antibody, or a lyophilized preparation of the antibody, and instructions for administering the composition to a patient in need thereof and/or for reconstituting the lyophilized product.

The present disclosure also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient, e.g., an above-described antibody, is sterile and suitable for administration as a particulate free solution. In certain aspects, the formulation is suitable for intravenous administration, such as for intravenous infusion to a human or animal.

In a specific aspect, the formulations of the disclosure are formulated in single dose vials as a sterile liquid. Exemplary containers include, but are not limited to, vials, bottles, pre-filled syringes, IV bags, and blister packs (comprising one or more pills). Optionally associated with such container(s) can be a notice in the form prescribed by a governmental agency regulating the manufacture, use or sale of pharmaceuticals or biological products, which notice reflects approval by the agency of manufacture, use or sale for human diagnosis and/or administration.

As with any pharmaceutical product, the packaging material and container are designed to protect the stability of the product during storage and shipment. Further, the products of the disclosure include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, etc., and other monitoring information.

A kit for diagnostic assays may comprise a solution containing an antibody, or antigen-binding fragment thereof, or a lyophilized preparation of an antibody or fragment thereof of the disclosure, wherein the antibody or fragment binds specifically to one or more targets, as well as reagents for detecting such antibodies. The antibody may be labeled according to methods known in the art and described herein, including but not limited to labels such as small molecule fluorescent tags, proteins such as biotin, GFP or other fluorescent proteins, or epitope sequences such as his or myc. Similarly, primary antibodies used for detecting the antibody may be included in the kit. Primary antibodies may be directed to sequences on the antibody or to labels, tags, or epitopes with which the antibodies are labeled. Primary antibodies may, in turn, be labeled for detection, or, if further amplification of the signal is desired, the primary antibodies may be detected by secondary antibodies, which may also be included in the kit.

Kits for research use are also contemplated. Such kits may, for example, resemble kits intended for diagnostic or therapeutic uses but further include a label specifying that the kit and its use is restricted to research purposes only.

G. Modification and/or Engineering

Another aspect of the disclosure includes the modification and/or engineering of the antibodies, bispecific antibodies, and/or antigen binding fragments thereof that are described herein. For example, antibodies, bispecific antibodies, and/or antigen binding fragments thereof include, without limitation, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual-specific antibody, and a bispecific antibody. Non-limiting embodiments of such constructs and antibody formats are illustrated in the Examples and throughout the disclosure. The disclosed antibodies, bispecific antibodies, and antigen binding fragments thereof, can also be used to generate fusion proteins and/or for antibody-targeted cell fusion, such as with immunological fusion partners and cells.

The disclosed antibodies, bispecific antibodies, and antigen binding fragments thereof, can also be used to produce therapeutic immunoconjugates, wherein the disclosed antibodies, bispecific antibodies, or antigen binding fragments thereof, are conjugated with one or more therapeutic agents. Non-limiting embodiments of such immunoconjugates are illustrated throughout the disclosure. In addition, for example, disclosed antibodies, bispecific antibodies, or antigen binding fragments thereof, can be used in the production of Antibody-Drug-Conjugates (ADC). Drugs that can be used include, without limitation, inhibitors of cell proliferation, anticancer agents, agents that induce apoptosis, and the like, and combinations thereof.

As discussed herein, the practice of the methods disclosed herein employs, unless otherwise indicated, available techniques of molecular biology (including recombinant techniques), microbiology, cell biology, biochemistry and immunology, which are familiar to the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991).

The following examples provide an illustration of some of the aspects and embodiments described above, and are not intended to limit the scope of the claimed invention.

### EXAMPLES

### Example 1. Affinity Maturation of m912 Antibody by Phage Display

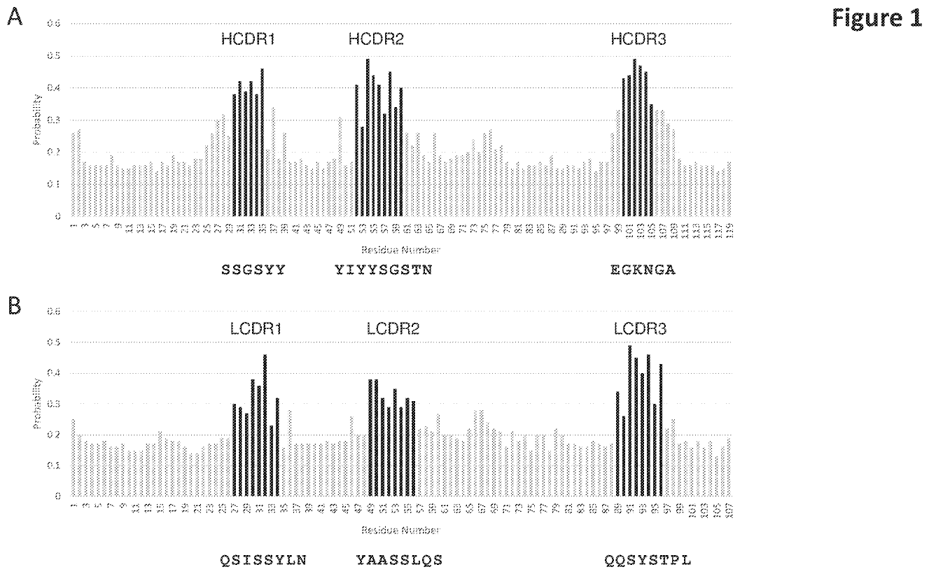

We began our affinity maturation campaign by defining CDR sequences of m912 variable regions. We submitted m912 VH and VL sequences (Table 1) to ProABC-2 webserver for prediction. ProABC-2 uses machine learning approaches to predict paratopes of an antibody[8]. Probabilities of residues involving antigen contacts via either hydrogen bonds, or hydrophobic interactions, or other non-bonded interactions were calculated and visualized (FIG. 1). The accuracy of the predicted CDR loops was confirmed by recently solved m912 structure [9].

TABLE 1
VH and VL sequences of MSLN antibody m912.

| | VH sequence | VL sequence |
|---|---|---|
| m912 | QVQLQESGPGLVKPSETLSLTCTVSGGSVSSGSY | DIQMTQSPSSLSASVGDRVTITCRASQSISSYL |
| | YWSWIRQPPGKGLEWIGYIYYSGSTNYNPSLKSR | NWYQQKPGKAPKLLIYAASSLQSGVPSRFSGSG |
| | VTISVDTSKNQFSLKLSSVTAADTAVYYCAREGK | SGTDFTLTISSLQPEDFATYYCQQSYSTPLTFG |
| | NGAFDIWGQGTMVTVSS | GGTKVEIK |
| | (SEQ ID NO. 1) | (SEQ ID NO. 2) |

A full-scale affinity maturation campaign was initiated by random mutagenesis of the predicted CDRs in m912's variable regions. A total of sixteen libraries were constructed with eight for HCDRs and eight for LCDRs (Table 2). The number of libraries for individual CDRs is dependent on the length of the predicted CDRs. The longer a CDR is, the more libraries are likely designated. Each library usually covers 4-5 residues. If a CDR requires two or more libraries to cover, these libraries will be pooled together for panning. Given the fact that HCDR3 plays a very important role in interacting with its cognate antigen, we put more emphasis on HCDR3 by constructing densely overlapped libraries. In the case of HCDR3, three overlapping libraries covering the six predicted residues were constructed (Table 2) and were panned independently. Depending on the panning results, additional libraries could be constructed to obtain the optimal consensus sequences.

TABLE 2
Library construction (oligonucleotides) for affinity maturation of m912 antibody.

| | | Target | Forward primer | Reverse primer |
|---|---|---|---|---|
| CDR | Library | sequence | (5′→3′) | (5′→3′) |
| HCDR1 | HP1-1 | SSGS | TCCGTCNNSNNSNNSNNSTACT | GCCACCAGAGACAGTGCAGGTG |
| SSGSYY | | (SEQ ID: | ACTGGAGCTGGATCC | (SEQ ID NO. 4) |
| (SEQ ID: | | 67) | (SEQ ID NO. 3) | |
| 61) | HP1-2 | SGSY | GTCAGCNNSNNSNNSNNSTACT | GGAGCCACCAGAGACAGTGCAG |
| | | (SEQ ID: | GGAGCTGGATCCGGC | (SEQ ID NO. 6) |
| | | 68) | (SEQ ID NO. 5) | |
| | HP1-3 | GSYY | AGCAGTNNSNNSNNSNNSTGGA | GACGGAGCCACCAGAGACAGEG |
| | | (SEQ ID: | GCTGGATCCGGCAGC | (SEQ ID NO. 8) |
| | | 69) | (SEQ ID NO. 7) | |
| HCDR2 | HP2-1 | SGSTN | TATTACNNSNNSNNSNNSNNST | GATATACCCAATCCACTCCAG |
| YIYYSGST | | (SEQ ID: | ACAACCCCTCCCTCAAGAG | (SEQ ID NO. 10) |
| N | | 70) | (SEQ ID NO. 9) | |
| (SEQ ID: | | | | |
| 62) | | | | |
| HCDR3 | HP3-1 | EGKN | gcgagaNNSNNSNNSNNSgggg | ACAGTAATACACGGCCGTGTC |
| EGKNGA | | (SEQ ID: | cttttgatatctgggg | (SEQ ID NO. 12) |
| (SEQ ID: | | 71) | (SEQ ID NO. 11) | |
| 63) | HP3-2 | GKNG | agagagNNSNNSNNSNNSgctt | CGCACAGTAATACACGGCCG |
| | | (SEQ ID: | ttgatatctggggcca | (SEQ ID NO. 14) |
| | | 72) | (SEQ ID NO. 13) | |
| | HP3-3 | KNGA | gaggggNNSNNSNNSNNStttg | TCTCGCACAGTAATACACGG |
| | | (SEQ ID: | atatctggggccaagg | (SEQ ID NO. 16) |
| | | 73) | (SEQ ID NO. 15) | |
| | HP3-4 | EGKNGA | gcgagaGASNNSNNSNNSNNSN | ACAGTAATACACGGCCGTGTC |
| | | (SEQ ID: | NStttgatatctggggccaagg | (SEQ ID NO. 18) |
| | | 74) | (SEQ ID NO. 17) | |
| LCDR1 | LP1-1 | ISSYL | CAGAGCNNSNNSNNSNNSNNSA | ACTTGCCCGGCAAGTGATGGTG |
| QSISSYLN | | (SEQ ID: | ATTGGTATCAGCAGAAAC | (SEQ ID NO. 20) |
| (SEQ ID: | | 75) | (SEQ ID NO. 19) | |
| 64) | | | | |
| LCDR2 | LP2-1 | YAAS | CTGATCNNSNNSNNSNNSAGTT | GAGCTTAGGGGCTTTCCCTGG |
| YAASSLQS | | (SEQ ID: | TGCAAAGTGGGGTCC | (SEQ ID NO. 22) |
| (SEQ ID: | | 76) | (SEQ ID NO. 21) | |
| 65) | LP2-2 | SLQS | GCATCCNNSNNSNNSNNSGGGG | AGCATAGATCAGGAGCTTAGG |
| | | (SEQ ID: | TCCCATCAAGGTTCAG | (SEQ ID NO. 24) |
| | | 77) | (SEQ ID NO. 23) | |
| LCDR3 | LP3-1 | QQSY | tactgtNNSNNSNNSNNSagta | GTAAGTTGCAAAATCTTCAG |
| QQSYSTPL | | (SEQ ID: | ccccgctcactttcggc | (SEQ ID NO. 26) |
| (SEQ ID: | | 78) | (SEQ ID NO. 25) | |
| 66) | LP3-2 | SYST | caacagNNSNNSNNSNNSccgc | ACAGTAGTAAGTTGCAAAATC |
| | | (SEQ ID: | tcactttcggcggaggg | (SEQ ID NO. 28) |
| | | 79) | (SEQ ID NO. 27) | |
| | LP3-3 | STPL | agttacNNSNNSNNSNNSactt | CTGTTGACAGTAGTAAGTTGC |
| | | (SEQ ID: | tcggcggagggaccaag | (SEQ ID NO. 30) |
| | | 80) | (SEQ ID NO. 29) | |
| | LP3-4 | PLTF | agtaccNNSNNSNNSNNSggcg | GTAACTCTGTTGACAGTAGTAA |
| | | (SEQ ID: | gagggaccaaggtggag | G |
| | | 81) | (SEQ ID NO. 31) | (SEQ ID NO. 32) |
| | LP3-5 | SYSTPL | CAACAGNNSTWCNNSNNSNNSN | ACAGTAGTAAGTTGCAAAATC |
| | | (SEQ ID: | NSACTTTCGGCGGAGGGACCAA | (SEQ ID NO. 34) |
| | | 82) | G | |
| | | | (SEQ ID NO. 33) | |

Libraries were constructed using a pair of 5′ phosphorylated oligonucleotides to amplify the entire plasmid (Table 2). The forward primer was synthesized as a degenerated oligonucleotide that contains 4-5 NNS codons to randomly mutagenize the targeted sequences. PCR was performed using Q5 High-Fidelity DNA polymerase (New England BioLabs, M0491L) for 30 cycles. The PCR product was digested by DpnI to remove template plasmid DNA, then extracted from 0.8% agarose gel (ThermoFisher, G501808). The purified PCR product was then self-ligated and electroporated into TG1 electrocompetent cells (Lucigen, 60502). Each library is required to achieve at least 100-fold coverage of the calculated complexity. For example, for a library covering 4 residues, the targeting complexity should be >100×20\hat{ }4 (i.e., 1.6×10\hat{ }7).

After electroporation, TG1 cells were spread onto a large panning plate (ThermoFisher, 240845) and grown in 30° C. incubator overnight. Next day, the bacterial lawn was scrapped and resuspended in 10 mL 2YT medium supplemented with 100 mg/mL carbenicillin 15% glycerol. A small fraction of bacterial suspension was inoculated into 100 mL 2YT medium plus carbenicillin and 2% glucose to OD₆₀₀~0.01. The culture was allowed to grow at 37° C. with shaking at 250 rpm until OD₆₀₀ reached ~0.5. The log-phase bacteria were infected with M13KO7 helper phage (ThermoFisher, 18311019), then grown at 30° C. overnight in the presence of kanamycin and carbenicillin. The phage library was precipitated from the collected supernatant using ⅕ volume of PEG solution which contains 20% PEG-8000 and 2.5 M NaCl.

Panning was performed in the solution format. Biotinylated mesothelin (Sino Biological, 13128-H01H-B50-B) was incubated with M-280 Streptavidin magnetic Dynabeads (ThermoFisher, 11205D) and a phage library. Two or three rounds of panning were carried out by against the deceasing concentration of mesothelin. The mesothelin concentration used in the first round of panning started from 20 nM, and 1/10 concentration of mesothelin from the previous round was used in the subsequent rounds. Unbound phages were washed away using PBST (0.1% Tween-20 in PBS). Bound phages were eluted using 0.1 N HCl and rescued using log-phase TG1 cells. TG1 cells were then spread at appropriate density to 2YT agar plates containing 100 mg/mL carbenicillin and 2% glucose so that single colonies could be identified and sequenced.

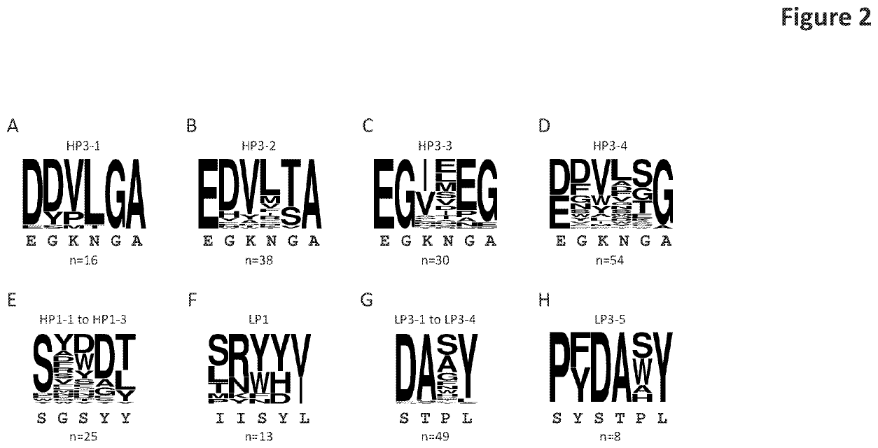

FIG. 2 summarizes panning results of the m912 campaign using WebLogo[10]. Panning against HCDR1, HCDR3, LCDR1, and LCDR3 but not against HCDR2 or LCDR2 resulted in sequence enrichment and consensus sequences. For CDRs covered by two or more libraries, panning was performed by pooling libraries together except for HCDR3. The three HCDR3 libraries were panned individually. From HP3-1 panning, the negatively charged aspartic acid (D) was enriched at position 1 and 2, while the hydrophobic residues valine (V) and leucine (L) were enriched at position 3 and 4 (FIG. 2A). This pattern was preserved in HP3-2 panning even though the glycine (G) at the fifth position was replaced by threonine (T) or serine (S) (FIG. 2B). For HP3-3 panning, the glutamic acid (E) and glycine (G) was enriched at position 5 and 6 (FIG. 2C). Based on the panning results, we can deduce that position 1 of HCDR3 prefers a negatively charged residue and position 6 tends to prefer glycine over alanine. Given the fact that HCDR3 only contains 6 residues, a fourth library (HP3-4) was constructed by assigning a negatively charged residue to the first position and by randomizing the last five residues (Table 1). After three rounds of panning, a consensus sequence was generated in which the glycine (G) was indeed predominantly enriched at position 6 whereas position 2-5 seemed less restrictive (FIG. 2D). With the HP3-4 library, we have thoroughly examined each position of HCDR3, therefore, we are confident that the sequences enriched from the HP3-4 library should contain the best optimal sequences for HCDR3.

LCDR3 was predicted to be the sequence from residue 89 to residue 98 of m912 VL using IMGT definition. However, LCDR3 could be at least two amino acids shorter than that based on proABC-2's prediction (FIG. 1B). To avoid potential off targeting, we constructed four libraries (LP3-1, LP3-2, LP-3, and LP3-4) to cover ten residues of broadly defined LCDR3, with each library randomized four residues. By panning, we found that LP3-3 was predominantly enriched from the pool of four libraries and a consensus sequence of DAXY was generated (FIG. 2G). The panning results suggest that the sequence of STPL targeted by the LP3-3 library is likely to be part of the hot spot for mutagenesis. To explore whether the two amino acids preceding STPL could be mutated, we constructed a fifth library (LP3-5) by randomizing the sequence of SYSTPL, but the position for tyrosine (Y) was only allowed to be either tyrosine (Y) or phenylalanine (F). Panning of this expanded library did not change the consensus sequence of DAXY, however, position 1 was preferred to be proline (P) (FIG. 2H). These results support proABC-2's prediction and demonstrate value of the tool for CDR prediction.

To rank candidate sequences from each panning, we prepared soluble scFv for ELISA analysis. The soluble scFv was prepared by inoculating single colonies to 100 ml 2YT+carbenicillin+2% glucose medium in a 96-well plate. To gauge the binding ability of candidates in comparison to the parental m912 antibody, we also inoculated three parental colonies on the same plate. The plate was allowed to shake at 250 rpm 37° C. overnight. Next morning, 5 mL overnight cultures were transferred into a new 96-well plate that contained 100 mL fresh 2YT+carbenicillin medium. After shaking at 37° C. for 2 hours, the plate was supplemented with additional 50 mL 2YT carbenicillin plus 3 mM IPTG and continued to incubate at 37° C. with shaking for 3-4 hours. The soluble scFv was released from periplasm by adding 50 mL 2YT carbenicillin medium containing 1000 units of polymyxin B sulfate (Sigma, P0972) and by incubating at 37° C. with shaking for an additional hour. The plate was centrifuged and the supernatants containing released scFv was ready for ELISA and Western blot.

The ELISA screening was performed as follows. The recombinant mesothelin (PeproTech, 100-63) at 1 mg/mL in PBS was coated to an Immulon 4 HBX plate (ThermoFisher, 3855) at 4° C. overnight. The plate was blocked with 4% dry milk in PBST (PBS+0.1% Tween) and then incubated with the 1:1 diluted scFv supernatant for 1 hour at room temperature. The bound scFv was detected using the anti-myc antibody (ThermoFisher, MA1-980) at 1 mg/mL and the secondary anti-mouse Fc antibody (R&D Systems, HAF007) at 0.5 mg/mL. The plate was washed three times with PBST between each incubation step. The plate was developed by addition of 1-step Ultra TMB-ELISA substrate (ThermoFisher, 34029). The color reaction was stopped by addition of 2 M sulfuric acid. OD₄₀₅ was read on SpectraMax M3 reader (Molecular Devices, CA, USA).

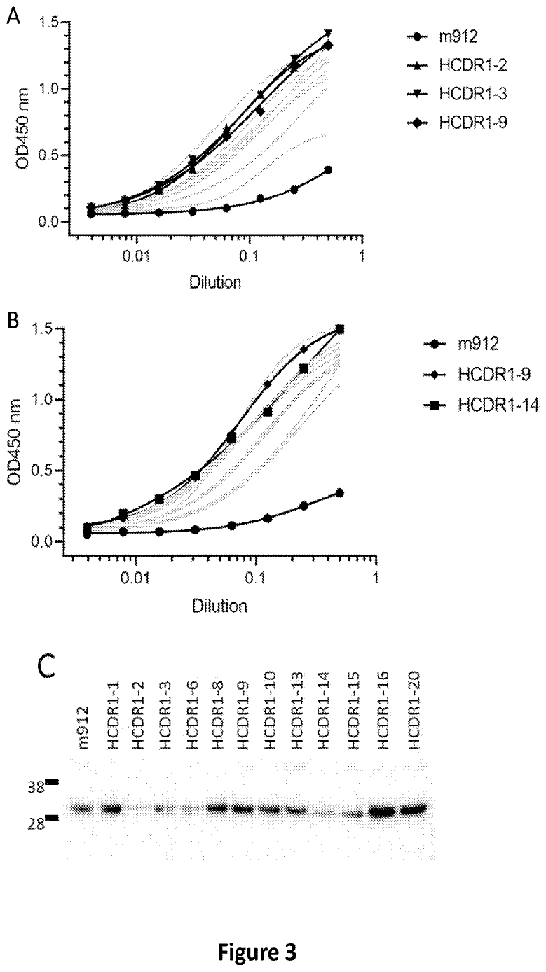

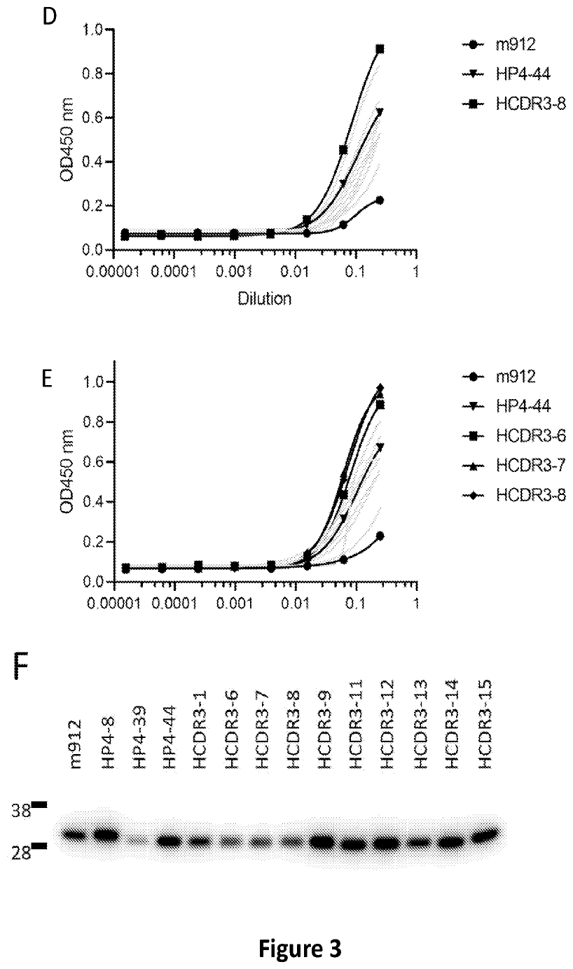

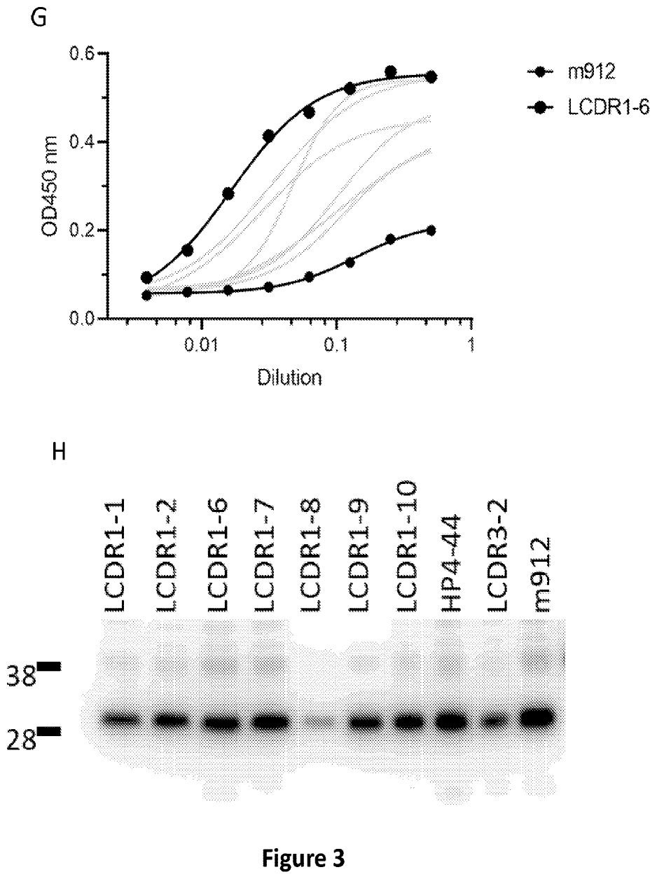

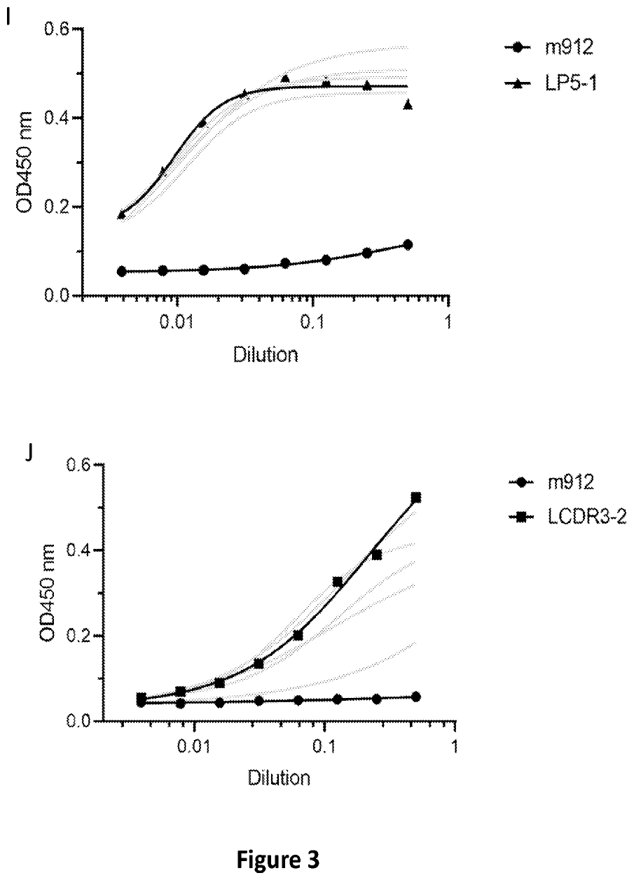

Despite most of colonies screened by scFv ELISA showed better binding than the parental m912, we generally selected 10-20 clones for further validation. The scFv ELISA was performed by applying serially diluted 1:2 scFv supernatants to the mesothelin-coated plate. The m912 scFv supernatant served as a control. We also performed Western blot to examine scFv at protein level. The soluble scFv was detected using a c-Myc monoclonal antibody (9E10) (ThermoFisher, 13-2500). We preferred to select clones with higher binding signals in scFv ELISA and lower signals on Western blot. As shown in FIG. 3, we selected four clones from twenty candidates for HCDR1 (FIG. 3A-3C), three clones from fourteen candidates for HCDR3 (FIG. 3D-3F), one clone from seven candidates for LCDR1 (FIGS. 3G and 3H), and two clones from 12 candidates for LCDR3 (FIGS. 31 and 3J) for making combo antibodies. In addition, two clones from HCDR3 panning with low to average binding activities were also included. The sequences of the selected clones are listed in Table 3 for further evaluation.

TABLE 3
Beneficial mutations identified by panning and
soluble scFv ELISA.

| CDR | Clone | Mutation |
|---|---|---|
| HCDR1 | HCDR1-2 | SSYHDL |
| | | (SEQ ID NO. 35) |
| | HCDR1-3 | SSRDDL |
| | | (SEQ ID NO. 36) |
| | HCDR1-9 | SSFTDT |
| | | (SEQ ID NO. 37) |
| | HCDR1-14 | SSYYST |
| | | (SEQ ID NO. 38) |
| HCDR3 | HP1-A13 | DDVLGA |
| | | (SEQ ID NO. 39) |
| | HP4-44 | EDVLSG |
| | | (SEQ ID NO. 40) |
| | HCDR3-6 | EDALTG |
| | | (SEQ ID NO. 41) |
| | HCDR3-7 | DFVLSG |
| | | (SEQ ID NO. 42) |
| | HCDR3-8 | DNVFTG |
| | | (SEQ ID NO. 43) |
| LCDR1 | LCDR1-6 | SRYYV |
| | | (SEQ ID NO. 44) |
| LCDR3 | LCDR3-2 | SYDASY |
| | | (SEQ ID NO. 45) |
| | LP2 | STDAFY |
| | | (SEQ ID NO. 46) |
| | LP5-1 | PYDASY |
| | | (SEQ ID NO. 47) |

### Example 2. Incorporation of Beneficial CDR Mutations into IgGs

The selected CDR sequences in Table 3 were incorporated back into heavy chain and light chain of IgG by subcloning into corresponding expression vectors. HP1-A13 and HP4-44 were selected for generating antibodies with nanomolar affinities as determined earlier. For generating antibodies with even higher affinities, four best clones from HCDR1 libraries (HCDR1-2, HCDR1-3, HCDR1-9, and HCDR1-14) were combined with three best clones from HCDR3 libraries (HCDR3-6, HCDR3-7, and HCDR3-8) by overlapping PCR to generate 12 HCDR combo constructs. Similarly, two clones from LCDR3 libraries (LCDR3-2 and LP5-1) were combined with one clone from LCDR1 library (LCDR1-6) by overlapping PCR to generate two LCDR combo constructs. Then the 12 HCDR combo constructs were cotransfected with two LCDR3 constructs and two LCDR1/LCDR3 combo constructs to generate 48 combo antibodies (Table 4). Transfection was performed using PEI transfection method on a 24 deep well plate (Axygen, P-DW-10ML-24-C) that contained 2.5 mL 293F cells (ThermoFisher, R79007).

TABLE 4
Antibodies (48) created by combining mutations of HCDRs and LCDRs.

| Combo | MSLN- | MSLN- | MSLN- | MSLN- | MSLN- | MSLN- | MSLN- | MSLN- | MSLN- | MSLN- | MSLN- | MSLN- |
|---|---|---|---|---|---|---|---|---|---|---|---|---|
| IgG | 1 | 2 | 3 | 4 | 5 | 6 | 7 | 8 | 9 | 10 | 11 | 12 |
| LCDR | NA | NA | NA | NA | NA | NA | NA | NA | NA | NA | NA | NA |
| 1 | | | | | | | | | | | | |
| LCDR | LCDR | LCDR | LCDR | LCDR | LCDR | LCDR | LCDR | LCDR | LCDR | LCDR | LCDR | LCDR |
| 3 | 3-2 | 3-2 | 3-2 | 3-2 | 3-2 | 3-2 | 3-2 | 3-2 | 3-2 | 3-2 | 3-2 | 3-2 |
| HCDR | HCDR | HCDR | HCDR | HCDR | HCDR | HCDR | HCDR | HCDR | HCDR | HCDR | HCDR | HCDR |
| 3 | 3-6 | 3-6 | 3-6 | 3-6 | 3-7 | 3-7 | 3-7 | 3-7 | 3-8 | 3-8 | 3-8 | 3-8 |
| HCDR | HCDR | HCDR | HCDR | HCDR | HCDR | HCDR | HCDR | HCDR | HCDR | HCDR | HCDR | HCDR |
| 1 | 1-2 | 1-3 | 1-9 | 1-14 | 1-2 | 1-3 | 1-9 | 1-14 | 1-2 | 1-3 | 1-9 | 1-14 |
| Titer | 90.1 | 69.2 | 76.4 | 106.4 | 91.3 | 83.9 | 74.1 | 83.1 | 84.5 | 53.7 | 84.2 | 81.3 |
| (mg/L) | | | | | | | | | | | | |
| NSB | 30 | 41 | 11 | 40 | 24 | 8 | 17 | 10 | 7 | 18 | 23 | 1 |
| Rank- | | | | | | | | | | | | |
| ing | | | | | | | | | | | | |
| Combo | MSLN- | MSLN- | MSLN- | MSLN- | MSLN- | MSLN- | MSLN- | MSLN- | MSLN- | MSLN- | MSLN- | MSLN- |
| IgG | 13 | 14 | 15 | 16 | 17 | 18 | 19 | 20 | 21 | 22 | 23 | 24 |
| LCDR | LCDR | LCDR | LCDR | LCDR | LCDR | LCDR | LCDR | LCDR | LCDR | LCDR | LCDR | LCDR |
| 1 | 1-6 | 1-6 | 1-6 | 1-6 | 1-6 | 1-6 | 1-6 | 1-6 | 1-6 | 1-6 | 1-6 | 1-6 |
| LCDR | LP5-1 | LP5-1 | LP5-1 | LP5-1 | LP5-1 | LP5-1 | LP5-1 | LP5-1 | LP5-1 | LP5-1 | LP5-1 | LP5-1 |
| 3 | | | | | | | | | | | | |
| HCDR | HCDR | HCDR | HCDR | HCDR | HCDR | HCDR | HCDR | HCDR | HCDR | HCDR | HCDR | HCDR |
| 3 | 3-6 | 3-6 | 3-6 | 3-6 | 3-7 | 3-7 | 3-7 | 3-7 | 3-8 | 3-8 | 3-8 | 3-8 |
| HCDR | HCDR | HCDR | HCDR | HCDR | HCDR | HCDR | HCDR | HCDR | HCDR | HCDR | HCDR | HCDR |
| 1 | 1-2 | 1-3 | 1-9 | 1-14 | 1-2 | 1-3 | 1-9 | 1-14 | 1-2 | 1-3 | 1-9 | 1-14 |
| Titer | 22.3 | 22.7 | 17.3 | 30.1 | 32.6 | 26.5 | 22.8 | 42.6 | 45.1 | 40.9 | 29.2 | 55.3 |
| (mg/L) | | | | | | | | | | | | |
| NSB | 15 | 27 | 34 | 21 | 22 | 13 | 5 | 12 | 33 | 26 | 16 | 2 |
| Rank- | | | | | | | | | | | | |
| ing | | | | | | | | | | | | |
| Combo | MSLN- | MSLN- | MSLN- | MSLN- | MSLN- | MSLN- | MSLN- | MSLN- | MSLN- | MSLN- | MSLN- | MSLN- |
| IgG | 25 | 26 | 27 | 28 | 29 | 30 | 31 | 32 | 33 | 34 | 35 | 36 |
| LCDR | NA | NA | NA | NA | NA | NA | NA | NA | NA | NA | NA | NA |
| 1 | | | | | | | | | | | | |
| LCDR | LP5-1 | LP5-1 | LP5-1 | LP5-1 | LP5-1 | LP5-1 | LP5-1 | LP5-1 | LP5-1 | LP5-1 | LP5-1 | LP5-1 |
| 3 | | | | | | | | | | | | |
| HCDR | HCDR | HCDR | HCDR | HCDR | HCDR | HCDR | HCDR | HCDR | HCDR | HCDR | HCDR | HCDR |
| | 3-6 | 3-6 | 3-6 | 3-6 | 3-7 | 3-7 | 3-7 | 3-7 | 3-8 | 3-8 | 3-8 | 3-8 |
| HCDR | HCDR | HCDR | HCDR | HCDR | HCDR | HCDR | HCDR | HCDR | HCDR | HCDR | HCDR | HCDR |
| 1 | 1-2 | 1-3 | 1-9 | 1-14 | 1-2 | 1-3 | 1-9 | 1-14 | 1-2 | 1-3 | 1-9 | 1-14 |
| Titer | 49.1 | 43.6 | 39.3 | 53.4 | 69.6 | 43.9 | 73.8 | 102.7 | 124.9 | 29.3 | 68.9 | 64 |
| (mg/L) | | | | | | | | | | | | |
| NSB | 9 | 47 | 39 | 46 | 19 | 45 | 29 | 32 | 43 | 44 | 31 | 6 |
| Rank- | | | | | | | | | | | | |
| ing | | | | | | | | | | | | |
| Combo | MSLN- | MSLN- | MSLN- | MSLN- | MSLN- | MSLN- | MSLN- | MSLN- | MSLN- | MSLN- | MSLN- | MSLN- |
| IgG | 37 | 38 | 39 | 40 | 41 | 42 | 43 | 44 | 45 | 46 | 47 | 48 |
| LCDR | LCDR | LCDR | LCDR | LCDR | LCDR | LCDR | LCDR | LCDR | LCDR | LCDR | LCDR | LCDR |
| | 1-6 | 1-6 | 1-6 | 1-6 | 1-6 | 1-6 | 1-6 | 1-6 | 1-6 | 1-6 | 1-6 | 1-6 |
| LCDR | LCDR | LCDR | LCDR | LCDR | LCDR | LCDR | LCDR | LCDR | LCDR | LCDR | LCDR | LCDR |
| 3 | 3-2 | 3-2 | 3-2 | 3-2 | 3-2 | 3-2 | 3-2 | 3-2 | 3-2 | 3-2 | 3-2 | 3-2 |
| HCDR | HCDR | HCDR | HCDR | HCDR | HCDR | HCDR | HCDR | HCDR | HCDR | HCDR | HCDR | HCDR |
| 3 | 3-6 | 3-6 | 3-6 | 3-6 | 3-7 | 3-7 | 3-7 | 3-7 | 3-8 | 3-8 | 3-8 | 3-8 |
| HCDR | HCDR | HCDR | HCDR | HCDR | HCDR | HCDR | HCDR | HCDR | HCDR | HCDR | HCDR | HCDR |
| 1 | 1-2 | 1-3 | 1-9 | 1-14 | 1-2 | 1-3 | 1-9 | 1-14 | 1-2 | 1-3 | 1-9 | 1-14 |
| Titer | 35.8 | 56.2 | 58.2 | 59.5 | 69.5 | 68.7 | 64 | 53 | 97.6 | 86.7 | 111.9 | 72.8 |
| (mg/L) | | | | | | | | | | | | |
| NSB | 25 | 35 | 36 | 48 | 20 | 42 | 3 | 14 | 28 | 37 | 38 | 4 |
| Rank- | | | | | | | | | | | | |
| ing | | | | | | | | | | | | |

Titers of antibodies from transient expression in 2.5 mL 293F cells were measured by Octet. Ranking of non-specific binding to H929 cells was based on x-means of peaks from flow cytometry with smaller number indicating higher non-specific binding.

### Example 3. Screening Higher Affinity Binders Based on Kinetic Analyses of Single Concentration of Antigen

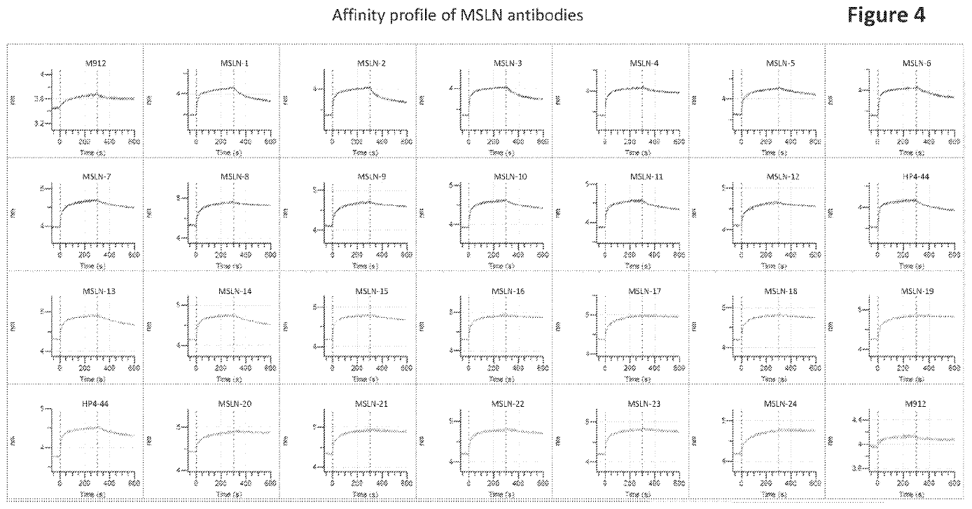

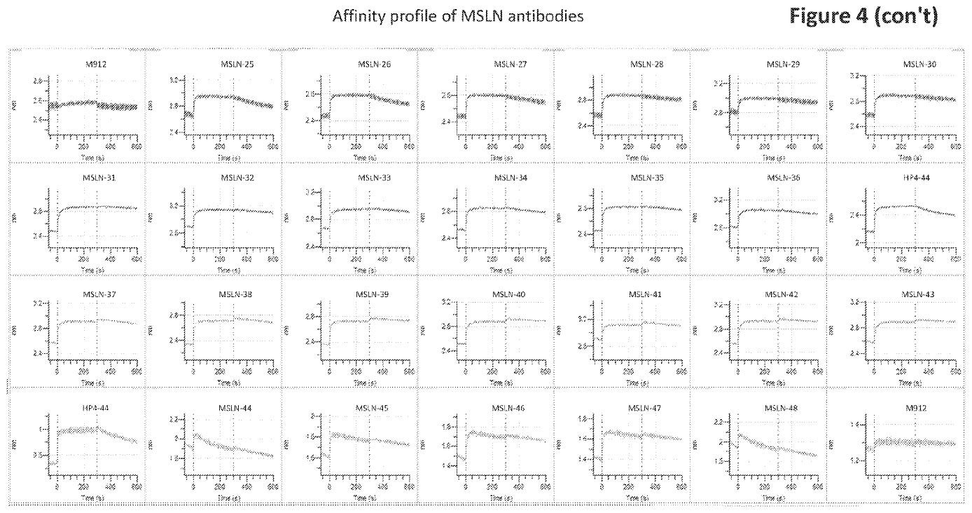

The day 6 culture media post transfection were harvested by centrigation and their expression titers were determined using Octet HTX (Forte Biosciences, TX, USA) (Table 3). To quickly scan binding profiles of these antibodies, we performed kinetic analyses using a single concentration of mesothelin at 125 nM in Octet. The kinetic experiments were performed by first capturing antibodies using anti-human Fc (AHC) biosensors followed by two baseline steps of 30 see each in the kinetic buffer. The antibody-captured biosensors were then submerged in wells containing 125 nM mesothelin for 5 min followed by 5 min of dissociation time in the kinetic buffer. The parental antibody m912 and two HCDR3 antibodies HP1-A13 and HP4-44 were included in the experiments. As shown in FIG. 4A-4H, most antibodies showed improved dissociation rates compared to the parental antibody m912.

### Example 4. Testing Non-Specific Binding on a MSLN-Negative Cell Line

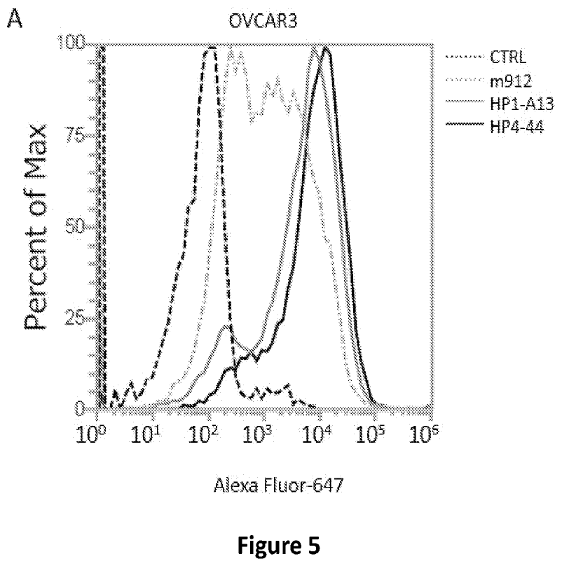

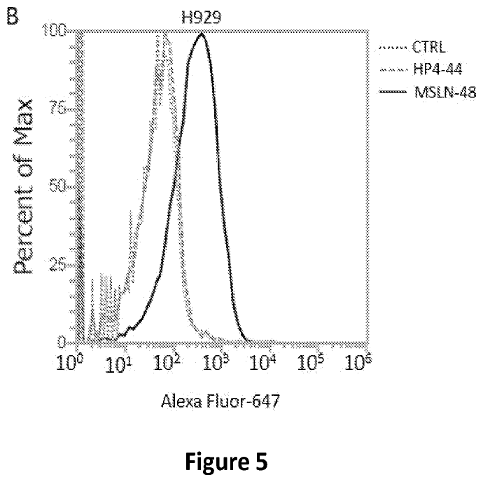

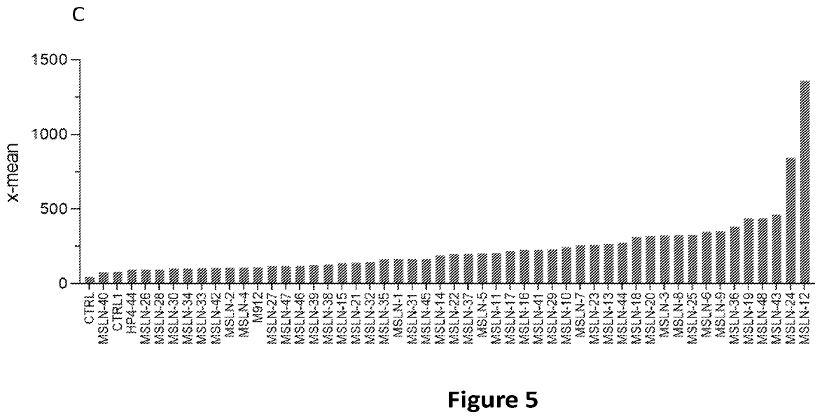

To explore whether these antibodies could have non-specific binding, we performed flow cytometry on a MSLN-negative cell line, NCI-H929 (ATCC, CRL-9068) using Attune NxT Flow Cytometer (ThermoFisher). H929 cells were collected from a suspension culture and were blocked by 1% FBS in PBS on ice for 1 hour. Approximately 3×10\hat{ }5 cells were incubated with 5 mg/mL antibody on ice for 1 hour, then subsequently washed and stained with Alexa Fluor®647 anti-human Fe (ThermoFisher, A21445) at 1:1000 dilution for 30 minutes. Cells were washed, stained with SYTOX Green (ThermoFisher, S7020) at 1:50000 dilution, and analyzed by flow cytometer. Negativity of H929 cells to mesothelin was confirmed using HP4-44 antibody that positively stained OVCAR3 cells (ATCC, HTB-161) (FIG. 5A) but negatively stained H929 cells (FIG. 5B). The 48 combo antibodies together with the parental m912 and HP4-44 were ranked for non-specific binding to H929 cells using peak x-means (FIG. 5C). Despite many antibodies showed no or low level of binding to H929 cells, a significant number of antibodies showed various degrees of binding to H929 cells, especially for those containing mutations from both HCDR1-14 and HCDR3-8 (FIGS. 5B and 5C, Table 4). Non-specific binding could be resulted from sticky paratopes of an antibody and/or due to aggregation property of an antibody. Nevertheless, the non-specific binding screening helped us to quickly eliminate inferior antibodies from a pool of combo antibodies.

### Example 5. Conversion of Leads into IgG-[L]-scFv^{CD3} Bispecific Format

A total of 11 combo antibodies were selected and converted to the IgG-[L]-scFv^{CD3} bispecific format (Table 5). The IgG-[L]-scFv format was first reported by Santich et al. (2020) in which a GD2 antibody fused with huOKT3-scFv to the C-terminus of the light chain showed superior antitumor activity both in vitro and in mice [11]. In this bispecific format, N297G and K322A double mutations were introduced in Fc to eliminate Fc receptor and complement binding activities, and a 3×G₄S linker was used to connect the light chain to huOKT3-scFv. The parental m912 and two antibodies (HP1-A13 and HP4-44) with a low-to-intermediate affinity from HCDR3 panning were also converted (Table 5).

TABLE 5
VH and VL sequences of a panel of antibodies selected for constructing bispecific antibodies in IgG-[L]-scFvCD3
format. CDR sequences are underlined and bold.

| Ab Ref. | VH sequence | VL sequence | HCDR1 | HCDR3 | LCDR1 | LCDR3 |
|---|---|---|---|---|---|---|
| m912 | [symbol] | [symbol] | SSGSYY | EGKNGA | QSISSYLN | QQSYSTFL |
| | [symbol] | [symbol] | (SEQ ID: | (SEQ ID: | (SEQ ID: | (SEQ ID: |
| | [symbol] | [symbol] | 61) | 63) | 64) | 66) |
| | [symbol] | [symbol] | | | | |
| | [symbol] | [symbol] | | | | |
| | [symbol] | (SEQ ID NO. 2) | | | | |
| | (SEQ ID NO. 1) | | | | | |
| HP1-A13 | [symbol] | [symbol] | SSGSYY | DDVLGA | QSISSYLN | QQSYSTFL |
| | [symbol] | [symbol] | (SEQ ID: | (SEQ ID | (SEQ ID: | (SEQ ID: |
| | [symbol] | [symbol] | 61) | NO. 39) | 64) | 66) |
| | [symbol] | [symbol] | | | | |
| | [symbol] | [symbol] | | | | |
| | [symbol] | (SEQ ID NO. 2) | | | | |
| | (SEQ ID NO. 48) | | | | | |
| HP4-44 | [symbol] | [symbol] | SSGSYY | EDVLSG | QSISSYLN | [symbol] |
| | [symbol] | [symbol] | (SEQ ID: | (SEQ ID | (SEQ ID: | (SEQ ID: |
| | [symbol] | [symbol] | 61) | NO. 40) | 64) | 46) |
| | [symbol] | [symbol] | | | | |
| | [symbol] | [symbol] | | | | |
| | [symbol] | (SEQ ID NO. 50) | | | | |
| | (SEQ ID NO. 49) | | | | | |
| MSLN-4 | [symbol] | [symbol] | SSYYST | EDALTG | QSISSYLN | SYDASY |
| | [symbol] | [symbol] | (SEQ ID | (SEQ ID | (SEQ ID: | (SEQ ID |
| | [symbol] | [symbol] | NO. 38) | NO. 41) | 64) | NO. 45) |
| | [symbol] | [symbol] | | | | |
| | [symbol] | [symbol] | | | | |
| | [symbol] | (SEQ ID NO. 52) | | | | |
| | (SEQ ID NO. 51) | | | | | |
| MSLN-21 | [symbol] | [symbol] | [symbol] | [symbol] | SRYYV | SYDASY |
| | [symbol] | [symbol] | (SEQ ID | (SEQ ID | (SEQ ID | (SEQ ID |
| | [symbol] | [symbol] | NO. 35) | NO. 43) | NO. 44) | NO. 45) |
| | [symbol] | [symbol] | | | | |
| | [symbol] | [symbol] | | | | |
| | [symbol] | (SEQ ID NO. 54) | | | | |
| | (SEQ ID NO. 53) | | | | | |
| MSLN-28 | [symbol] | [symbol] | SSYYST | [symbol] | QSISSYLN | [symbol] |
| | [symbol] | [symbol] | (SEQ ID | (SEQ ID | (SEQ ID: | (SEQ ID |
| | [symbol] | [symbol] | NO. 38) | NO. 41) | 64) | NO. 47) |
| | [symbol] | [symbol] | | | | |
| | [symbol] | [symbol] | | | | |
| | [symbol] | (SEQ ID NO. 55) | | | | |
| | (SEQ ID NO. 51) | | | | | |
| MSLN-30 | [symbol] | [symbol] | [symbol] | DFVLSG | QSISSYLN | PYDASY |
| | [symbol] | [symbol] | (SEQ ID | (SEQ ID | (SEQ ID: | (SEQ ID |
| | [symbol] | [symbol] | NO. 36) | NO. 42) | 64) | NO. 47) |
| | [symbol] | [symbol] | | | | |
| | [symbol] | [symbol] | | | | |
| | [symbol] | (SEQ ID NO. 55) | | | | |
| | (SEQ ID NO. 56) | | | | | |
| MSLN-31 | [symbol] | [symbol] | SSFTDT | DFVLSG | QSISSYLN | PYDASY |
| | [symbol] | [symbol] | (SEQ ID | (SEQ ID | (SEQ ID: | (SEQ ID |
| | [symbol] | [symbol] | NO. 37) | NO. 42) | 63) | NO. 46) |
| | [symbol] | [symbol] | | | | |
| | [symbol] | [symbol] | | | | |
| | [symbol] | (SEQ ID NO. 55) | | | | |
| | (SEQ ID NO. 57) | | | | | |
| MSLN-32 | [symbol] | [symbol] | SSGSYY | [symbol] | QSISSYLN | PYDASY |
| | [symbol] | [symbol] | (SEQ ID: | (SEQ ID | (SEQ ID: | (SEQ ID |
| | [symbol] | [symbol] | 61) | NO. 42) | 64) | NO. 47) |
| | [symbol] | [symbol] | | | | |
| | [symbol] | [symbol] | | | | |
| | [symbol] | (SEQ ID NO. 55) | | | | |
| | (SEQ ID NO. 58) | | | | | |
| MSLN-33 | [symbol] | [symbol] | [symbol] | [symbol] | QSISSYLN | [symbol] |
| | [symbol] | [symbol] | (SEQ ID | (SEQ ID | (SEQ ID: | (SEQ ID |
| | [symbol] | [symbol] | NO. 35) | NO. 43) | 64) | NO. 45) |
| | [symbol] | [symbol] | | | | |
| | [symbol] | [symbol] | | | | |
| | [symbol] | (SEQ ID NO. 55) | | | | |
| | (SEQ ID NO. 53) | | | | | |
| MSLN-33 | [symbol] | [symbol] | SSFTDT | [symbol] | SRYYV | SYDASY |
| | [symbol] | [symbol] | (SEQ ID | (SEQ ID | (SEQ ID | (SEQ ID |
| | [symbol] | [symbol] | NO. 37) | NO. 41) | NO. 44) | NO. 45) |
| | [symbol] | [symbol] | | | | |
| | [symbol] | [symbol] | | | | |
| | [symbol] | (SEQ ID NO. 60) | | | | |
| | (SEQ ID NO. 59) | | | | | |
| MSLN-40 | [symbol] | [symbol] | SSYYST | EDALTG | SRYYV | SYDASY |
| | [symbol] | [symbol] | (SEQ ID | (SEQ ID | (SEQ ID | (SEQ ID |
| | [symbol] | [symbol] | NO. 38) | NO. 41) | NO. 44) | NO. 45) |
| | [symbol] | [symbol] | | | | |
| | [symbol] | [symbol] | | | | |
| | [symbol] | (SEQ ID NO. 60) | | | | |
| | (SEQ ID NO. 51) | | | | | |
| MSLN-42 | [symbol] | [symbol] | SSRDDL | DFVLSG | SRYYV | SYDASY |
| | [symbol] | [symbol] | (SEQ ID | (SEQ ID | (SEQ ID | (SEQ ID |
| | [symbol] | [symbol] | NO. 36) | NO. 42) | NO. 44) | NO. 45) |
| | [symbol] | [symbol] | | | | |
| | [symbol] | [symbol] | | | | |
| | [symbol] | (SEQ ID NO. 60) | | | | |
| | (SEQ ID NO. 56) | | | | | |
| MSLN-45 | [symbol] | [symbol] | [symbol] | DNVFTG | SRYYV | SYDASY |
| | [symbol] | [symbol] | (SEQ ID | (SEQ ID | (SEQ ID | (SEQ ID |
| | [symbol] | [symbol] | NO. 35) | NO. 43) | NO. 44) | NO. 45) |
| | [symbol] | [symbol] | | | | |
| | [symbol] | [symbol] | | | | |
| | [symbol] | (SEQ ID NO. 60) | | | | |
| | (SEQ ID NO. 53) | | | | | |

[symbol]

indicates data missing or illegible when filed

The resulting constructs were transfected into 125 mL 293F cultures, and their expression titers were determined at day 6 post PEI transfection (Table 6).

TABLE 6
Titers of MSLN-[L]-scFv^{CD3} bsAbs expressed in 125 ml of
freestyle 293f cell. Titer were determined using Octet.

| bsAb | Titer (mg/L) |
|---|---|
| m912-[L]-scFv^{CD3} | 36.4 |
| HP1-A13-scFv^{CD3} | 43.3 |
| HP4-44-[L]-scFv^{CD3} | 39.2 |
| MSLN-4-[L]-scFv^{CD3} | 73.2 |
| MSLN-21-[L]-scFv^{CD3} | 16.8 |
| MSLN-28-[L]-scFv^{CD3} | 50.1 |
| MSLN-30-[L]-scFv^{CD3} | 30.5 |
| MSLN-31-[L]-scFv^{CD3} | 38.0 |
| MSLN-32-[L]-scFv^{CD3} | 36.5 |
| MSLN-33-[L]-scFv^{CD3} | 46.4 |
| MSLN-39-[L]-scFv^{CD3} | 8.1 |
| MSLN-40-[L]-scFv^{CD3} | 36.4 |
| MSLN-42-[L]-scFv^{CD3} | 12.2 |
| MSLN-45-[L]-scFv^{CD3} | 16.6 |

### Example 6. Binding Affinities of MSLN-[L]-scFv^{CD3} bsAbs

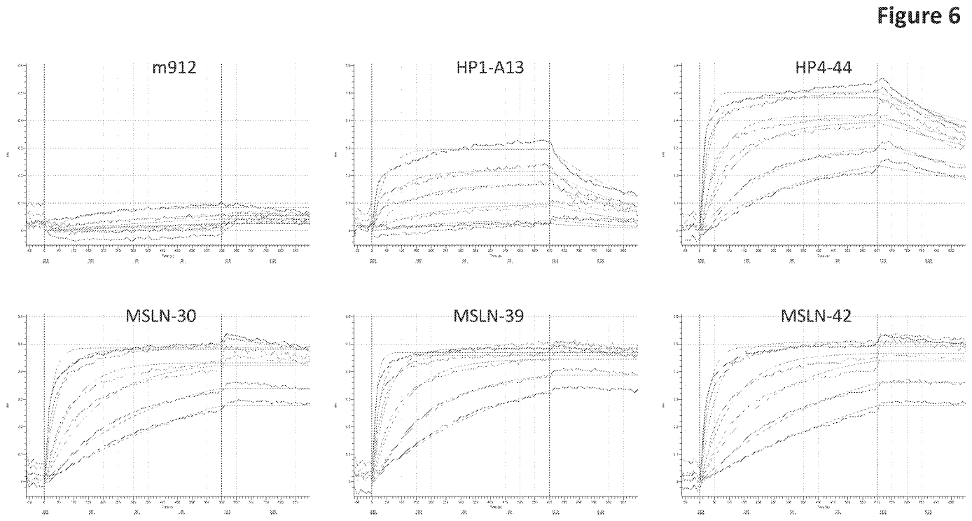

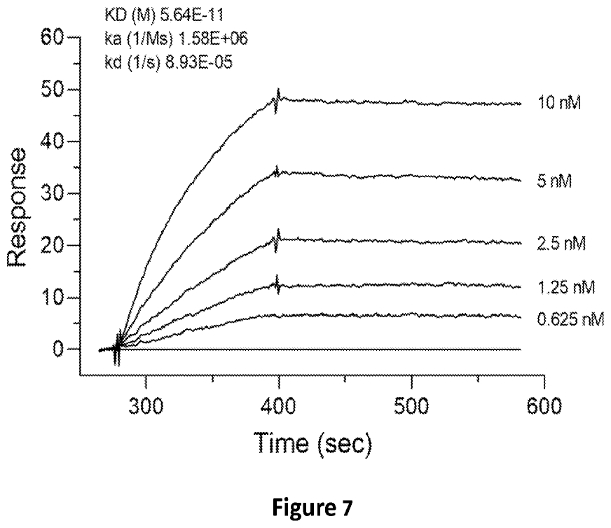

Binding affinities of the MSLN-[L]-scFv^{CD3} bsAbs were evaluated using Octet as described earlier. As shown in FIG. 6A-C, affinity of m912 was too low to be estimated. The dissociation constants (K_{D}S) for HP1-A13 bsAb and HP4-44 bsAB were estimated to be 28.7 nM and 2.9 nM, respectively, using a 1:1 binding model. K_{D}S for MSLN-30, MSLN-39, and MSLN-42 bsAbs were estimated to be 1-2 pM (Table 7), but these estimations might not be accurate because of Octet's detection limits. Therefore, one of our leads (MSLN-39) in IgG format was measured by Surface Plasmon Resonance (SPR) using Biacore 3000 (GE Healthcare). The MSLN-39 antibody was immobilized onto a sensor chip via a Fc antibody, then mesothelin was added to the sensor chip sequentially in five concentrations between 0.625 nM and 10 nM. Data were collected and globally fitted to a four-parameter non-linear curve fit model. As shown in FIG. 7, K_{D} of MSLN-39 IgG was determined to be 56.4 pM.

TABLE 7
Summary of parameters from kinetic experiments of selected
MSLN-[L]-scFv^{CD3} bsAbs by Octet.

| BsAb | KD (M) | Ka (1/Ms) | Kd (1/s) |
|---|---|---|---|
| m912-[L]-scFv^{CD3} | NA | NA | NA |
| HP1-A13-[L]-scFv^{CD3} | 2.87E−08 | 1.04E+05 | 2.97E−03 |
| HP4-44-[L]-scFv^{CD3} | 2.91E−09 | 2.78E+05 | 8.09E−04 |
| MSLN-30-[L]-scFv^{CD3} | 1.71E−12 | 2.21E+05 | 2.44E−07 |
| MSLN-31-[L]-scFv^{CD3} | 1.18E−12 | 2.58E+05 | 3.03E−07 |
| MSLN-39-[L]-scFv^{CD3} | 1.07E−12 | 3.79E+05 | 4.05E−07 |
| MSLN-42-[L]-scFv^{CD3} | 1.12E−12 | 1.75E+05 | 1.96E−07 |

(NA) Signals for m912-[L]-scFv^{CD3} bsAb were too weak to determine binding affinity.

### Example 7. Cell surface binding of MSLN-[L]-scFv^{CD3} bsAbs

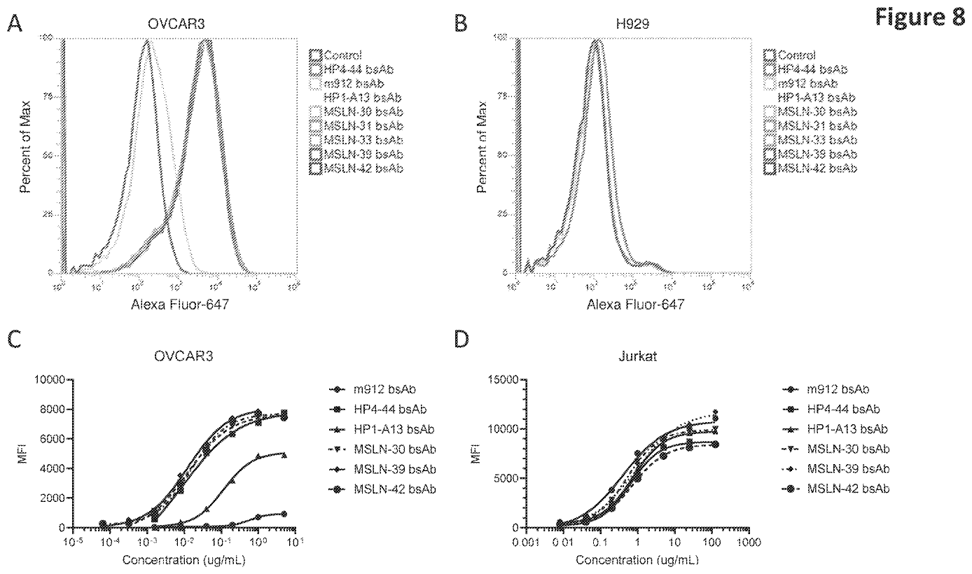

Flow cytometry was performed to evaluate cell surface binding of MSLN-[L]-scFv^{CD3} bsAbs on a mesothelin positive cell line OVCAR3. Once again, all our bsAbs specifically bound to OVCAR3 cells but not to the control H929 cells (FIGS. 8A and 8B). The binding was dose-dependent (FIG. 8C) and all bsAbs tested showed better binding than the parental m912 bsAb (EC₅₀ of 572 ng/mL). HP1-A13 bsAb with a K_{D} of 28.7 nM showed moderate binding with an EC₅₀ of 116 ng/mL. Other affinity-improved bsAbs including HP4-44 bsAb showed better binding to OVCAR3 cells with EC₅₀s between 9.6-15.7 ng/mL (Table 8). We also measured binding of our bsAbs to Jurkat cells (FIG. 8D). As expected, the overall bindings of our bsAbs to CD3 were similar to each other but much weaker than to MSLN (Table 8).

TABLE 8
Binding parameters of MSLN-[L]-scFv^{CD3} bsAbs to cell surface
of OVCAR3 and Jurkat cells by flow cytometry.

| | EC₅₀ (ng/mL) | EC₅₀ (ng/mL) |
|---|---|---|
| BsAb | OVCAR3 | Jurkat |
| M912-[L]-scFv^{CD3} | 572.1 | 403.2 |
| HP1-A13-[L]-scFv^{CD3} | 116.0 | 735.3 |
| HP4-44-[L]-scFv^{CD3} | 9.6 | 636.0 |
| MSLN-30-[L]-scFv^{CD3} | 12.1 | 521.2 |
| MSLN-39-[L]-scFv^{CD3} | 12.4 | 892.7 |
| MSLN-42-[L]-scFv^{CD3} | 15.7 | 730.0 |

### Example 8. Simultaneous Binding of MSLN-[L]-scFv^{CD3} bsAbs to Mesothelin and CD3

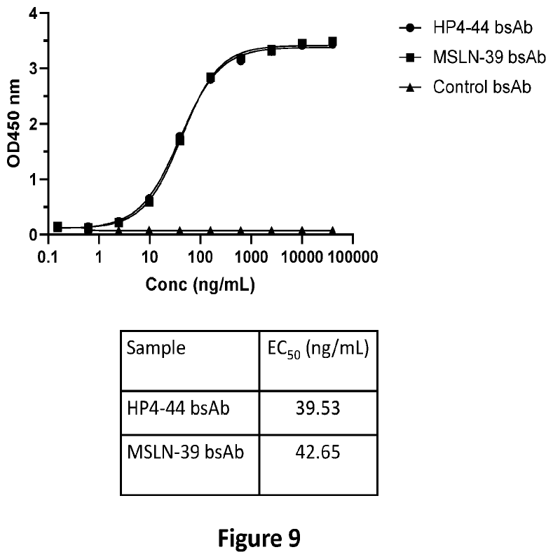

To detect simultaneous binding of mesothelin and CD3 by MSLN-[L]-scFv^{CD3} bsAbs, we developed a sandwich ELISA assay in which CD3e and CD3d heterodimer (ACRO Biosystems, Cat. No. CDD-H52Wa) was coated on the plate for capturing MSLN-[L]-scFv^{CD3} bsAbs with the CD3 arm. By supplementing a biotinylated MSLN (with Avi-tag from R&D Systems, Cat. No. AVI10679-050), we were able to detect simultaneous binding of MSLN to the MSLN arm using HRP-conjugated streptavidin (Jackson Immuno Research, Cat. No. 016-030-084). Two samples of MSLN-[L]-scFv^{CD3} bsAbs (HP4-44 and MSLN-39) tested in the assay showed simultaneous binding of antigens with similar EC₅₀s (FIG. 9).

### Example 9. In Vitro T Cell Activation by MSLN-[L]-scFv^{CD3} bsAbs

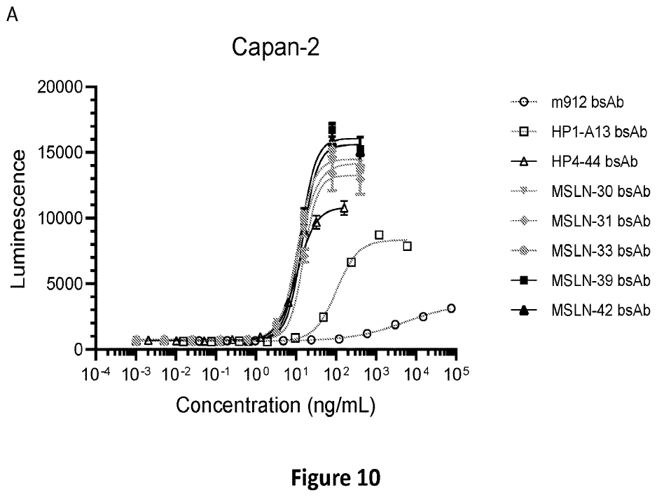

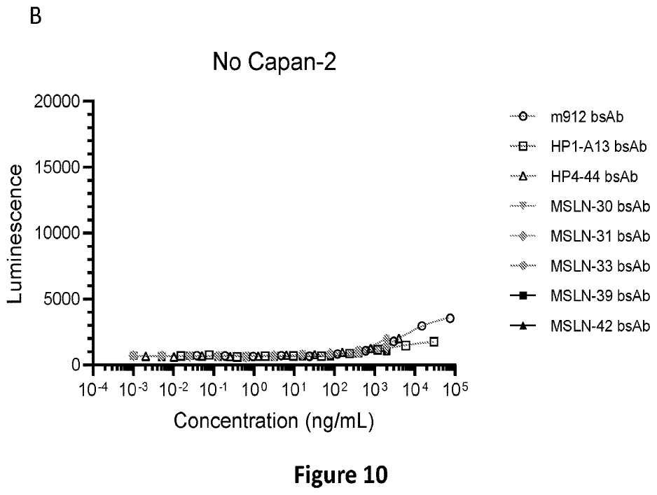

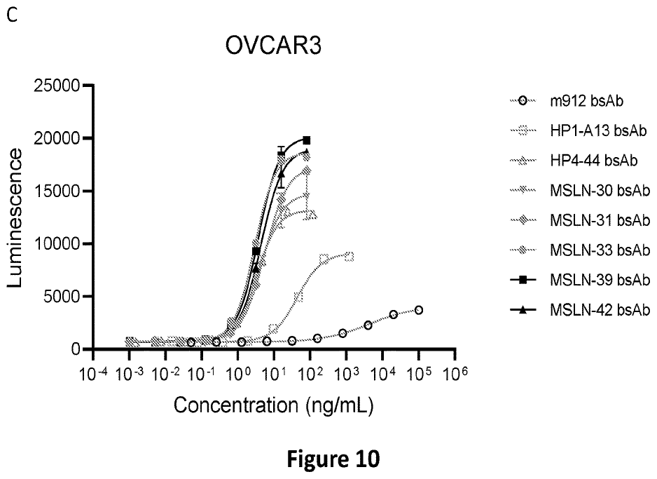

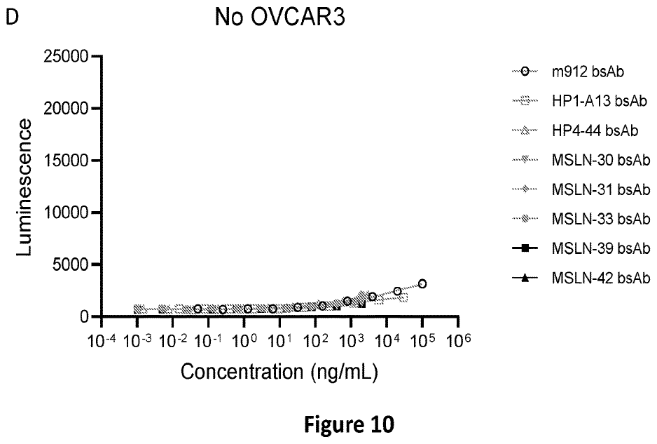

To investigate the MSLN-specific T-cell activation by MSLN-[L]-scFv^{CD3} bsAbs, we used a genetically engineered Jurkat cell line that expresses a luciferase reporter driven by an NFAT-response element. When these cells were exposed to an anti-TCR/CD3 stimulus, receptor-mediated signaling could induce luminescence via activation of nuclear transcription factors. The assay was performed by incubating Jurkat cells with MSLN-[L]-scFv^{CD3} bsAbs in the absence and presence of either OVCAR3 cells or Capan-2 cells. As shown in FIG. 10, MSLN-[L]-scFv^{CD3} bsAbs significantly induced T-cell activation in a dose-dependent manner (FIGS. 10A and 10C), whereas little activation was observed in the absence of target cells (FIGS. 10B and 10D). The potency observed for our bsAbs seems correlate well with its affinity. With a K_{D} of 72 nM, m912 bsAb showed little activity for T cell activation. With a K_{D} of 28.7 nM, HP1-A13 bsAb showed moderate activity with an EC₅₀ of 102 ng/mL and 109 ng/mL for Capan-2 and OVCAR3, respectively. With affinity around 3 nM, EC₅₀ for HP4-44 bsAb improved dramatically to 10 ng/mL (Table 9). Further improvements in affinity showed little improvement on EC₅₀. Instead, B max was significantly improved (FIGS. 10A and 10C). A possible explanation is that the accessibility of epitopes on target cells are limited which may result in saturated binding. The improvement in B max of T-cell activation is probably due to improvement in dissociation constant. Our bispecific molecules may hold T cells and target cells together with a longer time, thereby increasing the maximal activation.

TABLE 9
Efficacy of MSLN-[L]-scFv^{CD3} bsAbs measured by T cell
activation assay.

| | EC₅₀ (ng/mL) | EC₅₀ (ng/ml) |
|---|---|---|
| BsAb | OVCAR3 | CAPAN2 |
| M912-[L]-scFv^{CD3} | 6637.0 | 6917.0 |
| HP1-A13-[L]-scFv^{CD3} | 109.3 | 102.1 |
| HP4-44-[L]-scFv^{CD3} | 8.7 | 10.5 |
| MSLN-30-[L]-scFv^{CD3} | 9.8 | 11.7 |
| MSLN-31-[L]-scFv^{CD3} | 11.6 | 15.6 |
| MSLN-33-[L]-scFv^{CD3} | 6.7 | 10.7 |
| MSLN-39-[L]-scFv^{CD3} | 8.2 | 12.8 |
| MSLN-42-[L]-scFv^{CD3} | 9.3 | 14.4 |

### Example 10. Analyses of MSLN-[L]-scFv^{CD3} bsAbs Using KILR Cytotoxicity Assay

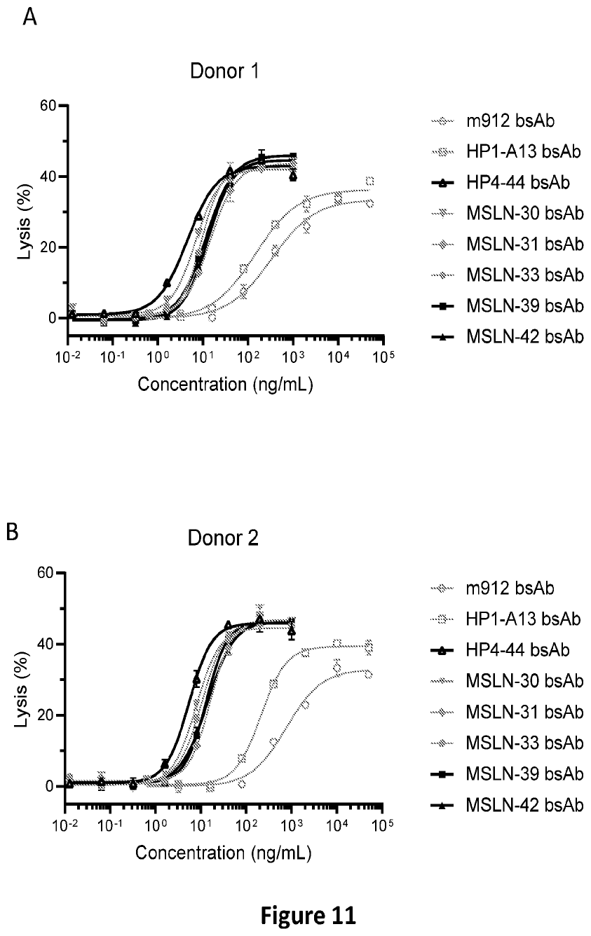

The same set of MSLN-[L]-scFv^{CD3} bsAbs was further analyzed by KILR cytotoxicity assay in which target Capan-2/KILR cells were incubated with PBMCs in the presence of our bsAbs. The target Capan-2/KILR cell line was generated by stable transfection of a KILR gene that expresses ePL-tagged reporter protein (DiscoverX). Once cells are killed, luminescence will be generated from released e-PL in the presence of KILR detection reagent. As shown in FIG. 11, HP4-44, MSLN-30, MSLN-31, MSLN-33, MSLN-39, and MSLN-42 bsAbs showed similar potency with EC₅₀s close to those from the T cell activation assays (Table 9). HP1-A13 bsAb, which is less potent in the T cell activation assay, was also less potent in KILR assays with a moderate EC₅₀. EC₅₀ for m912 seemed better in KILR assays than in T cell activation assays. The experiments using PBMCs from two donors produced comparable results (Table 10).

TABLE 10
Efficacy of MSLN-[L]-scFv^{CD3} bsAbs measured by KILR
cytotoxicity assay.

| | EC₅₀ (ng/ml) | EC₅₀ (ng/ml) |
|---|---|---|
| BsAb | Donor 1 | Donor 2 |
| M912-[L]-scFv^{CD3} | 330.9 | 762.7 |
| HP1-A13-[L]-scFv^{CD3} | 144.6 | 211.9 |
| HP4-44-[L]-scFv^{CD3} | 4.5 | 5.5 |
| MSLN-30-[L]-scFv^{CD3} | 6.8 | 7.8 |
| MSLN-31-[L]-scFv^{CD3} | 14.6 | 16.3 |
| MSLN-33-[L]-scFv^{CD3} | 9.2 | 10.7 |
| MSLN-39-[L]-scFv^{CD3} | 11.7 | 13.0 |
| MSLN-42-[L]-scFv^{CD3} | 12.7 | 14.0 |

### Example 11. Analyses of MSLN-[L]-scFv^{CD3} bsAbs Using Luciferase-Based Cytotoxicity Assay

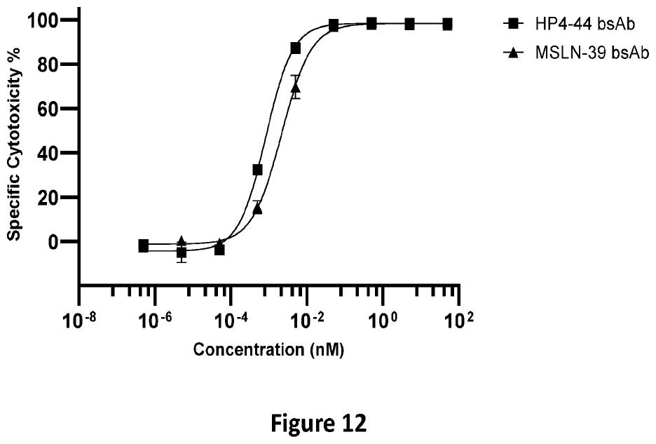

The cytotoxicity effect of MSLN-[L]-scFv^{CD3} bsAbs was also evaluated on gastric cancer cell line NCI-N87 in which the firefly luciferase was constitutively expressed. T cell-directed killing was measured by quantifying viability of target cells which is directly correlated with luciferase activity [12]. As shown in FIG. 12, two samples of MSLN-[L]-scFv^{CD3} bsAbs (HP4-44 and MSLN-39) tested in the assay showed similar activities, which is consistent with results from T cell activation assay in Example 9 and KILR cytotoxicity assay in Example 10.

### Example 12. Influence of Soluble Mesothelin on MSLN-[L]-scFv^{CD3} bsAbs

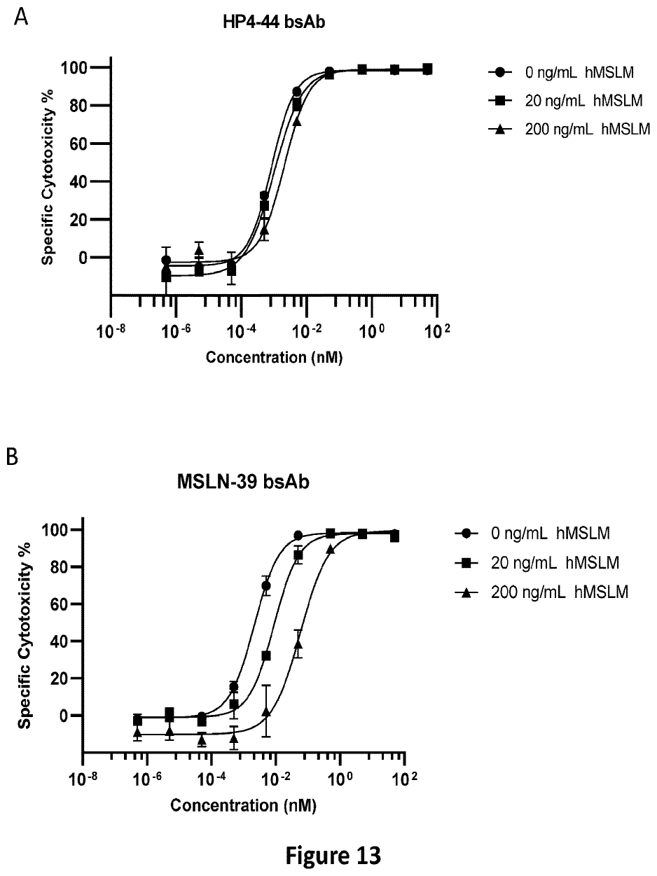

Mesothelin undergoes constant shedding from the surface of cancer cells and generates a pool of soluble protein in pleural fluid, ascites, and in blood. Serum MSLN could serve as a decoy receptor to neutralize antibody drugs[13]. To evaluate the impact of soluble MSLN on MSLN-[L]-scFv^{CD3} bsAbs, we employed luciferase-based cytotoxicity assay in the presence of soluble MSLN. As shown in FIG. 13, we observed that HP4-44 bsAb was less sensitive to soluble MSLN compared to MSLN-39. At 200 ng/mL of soluble MSLN, the IC₅₀ value of MSLN-39 bsAb increased by 27-fold compared to only 2.5-fold increase of HP4-44 bsAb. These results are consistent with the notion that high affinity antibodies are more susceptible to soluble antigens.

### Example 13. In Vivo Antitumor Effects of MSLN-[L]-scFv^{CD3} bsAbs in a Co-Grafting Model

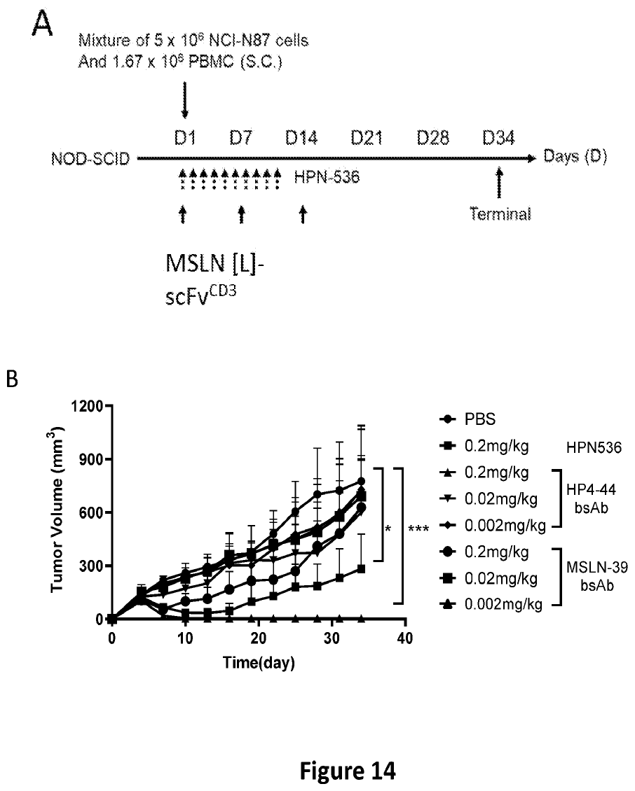

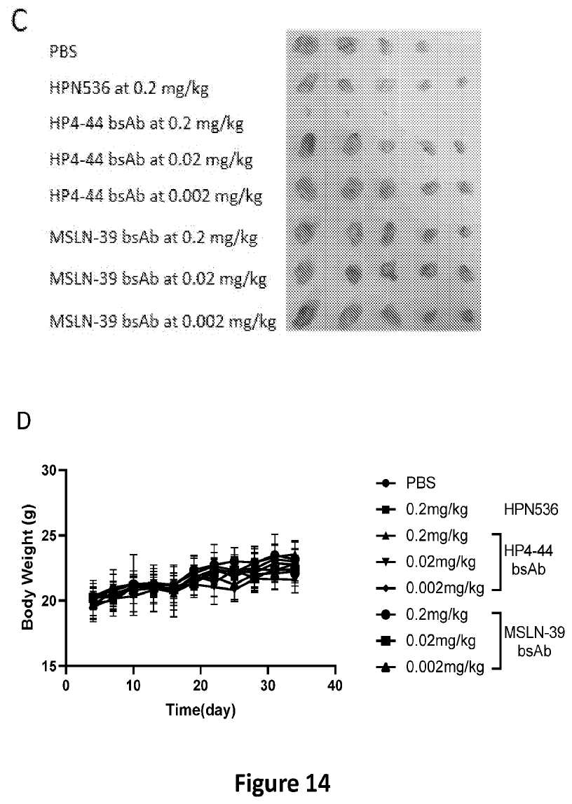

In vivo antitumor activity was evaluated in NOG-SCID mice with NCI-N87/PBMC co-grafting model. PBMC (1.67×10⁶) and human gastric cancer cells NCI-N87 (5×10⁶) were premixed and implanted s.c. in female NOD/SCID mice (n=5/group). Two samples of MSLN-[L]-scFv^{CD3} bsAbs (HP4-44 and MSLN-39) together with a positive control HPN536 were tested. HPN536 is a bispecific molecule developed by Harpoon Inc. in which four scFv domains were tandemly connected and were capable of binding to MSLN, HSA, and CD3. Mice were treated i.v. with vehicle control (PBS), HPN536 (0.2 mg/kg), or MSLN-[L]-scFv^{CD3} bsAbs at three different dose levels (0.002, 0.02 and 0.2 mg/kg) according to the scheme shown in FIG. 14A. HPN536 was subjected to 10 injections on the daily basis due to the short half-life. One mouse in the PBS group was too sick to be included in the analysis. Compared to PBS, both HPN536 and HP4-44 bsAb at 0.2 mg/kg exhibited significant inhibitory effect on tumor growth, whereas MSLN-39 bsAb, even though with better affinity, only exhibited moderate inhibition (FIGS. 14B and 14C). Shrinkage of tumor volume in the HP-44 bsAb-treated group was more pronounced at a dose of 0.2 mg/kg. In one mouse of this group, the tumor was completely abolished (FIG. 14C). Body weights of the mice did not differ between the treated groups and the control group (FIG. 14D).

### Example 14. Pharmacokinetics of MSLN-[L]-scFv^{CD3} bsAbs in Mice

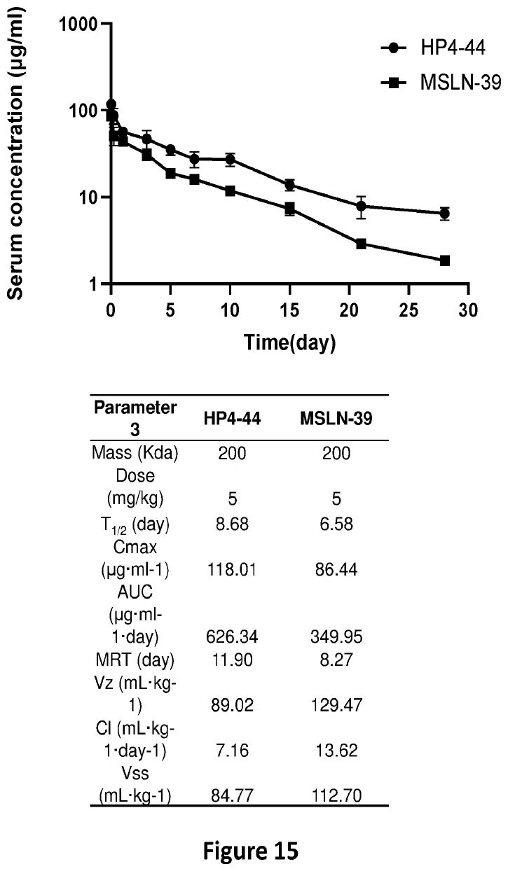

Single-dose pharmacokinetic studies were performed in BALB/c male mice at a dose of 5 mg/kg via intravenous injection. As shown in FIG. 15, both HP4-44 and MSLN-39 bsAbs showed a favorable PK profile with half-life (t½) of 8.68 days and 6.58 days, respectively. The maximum serum concentration (Cmax) of HP4-44 bsAb was 118.01 mg/mL, and the Cmax of MSLN-39 bsAb was 86.44 mg/mL. The area under the curve (AUC, a measure of systemic exposure) and total clearance (CL) of HP4-44 bsAb were 626.34 μg·day/mL and 7.16 mL/day/kg, respectively, whereas AUC and CL of MSLN-39 bsAb were 349.95 μg·day/mL and 13.62 mL/day/kg, respectively. The shorter half-life of MSLN-39 bsAb is probably related to quicker clearance of the drug in the body. The smaller steady-state volume of distribution (Vss) of HP4-44 bsAb suggested that it may have lower tissue penetration than MSLN-39 bsAb. Together, these results suggest that HP4-44 bsAb has better clearance and systemic exposure profiles in mice than MSLN-39 bsAb.

As the disclosure and illustrative examples demonstrate, bispecific antibodies comprising a region that specifically binds mesothelin can be used to target and fine-tune therapeutic interventions that relate to mesothelin-associated diseases or conditions. The examples illustrate example embodiments comprising a bispecific antibody that includes an anti-CD3 scFv connected to the C-terminus of a light chain variable region of a mesothelin antibody. The bispecific antibodies exemplified herein demonstrate excellent anti-tumor activity in vitro and in co-grafted mice models of disease. The bispecific antibodies exemplified herein demonstrate that binding of the constructs may be less sensitive to soluble mesothelin. Further, the bispecific antibodies exemplified herein demonstrate an excellent half life in vivo in animal models.

### REFERENCES

1. Pastan, I. and R. Hassan, Discovery of mesothelin and exploiting it as a target for immunotherapy. Cancer Res, 2014. 74(11): p. 2907-12.
2. Molloy, M. E., et al., Preclinical Characterization of HPN536, a Trispecific, T-Cell-Activating Protein Construct for the Treatment of Mesothelin-Expressing Solid Tumors. Clin Cancer Res, 2021. 27(5): p. 1452-1462.
3. Golfier, S., et al., Anetumab ravtansine: a novel mesothelin-targeting antibody-drug conjugate cures tumors with heterogeneous target expression favored by bystander effect. Mol Cancer Ther, 2014. 13(6): p. 1537-48.
4. Hassan, R., et al., Phase I clinical trial of the chimeric anti-mesothelin monoclonal antibody MORAb-009 in patients with mesothelin-expressing cancers. Clin Cancer Res, 2010. 16(24): p. 6132-8.
5. Hassan, R., et al., Phase 1 study of the immunotoxin LMB-100 inpatients with mesothelioma and other solid tumors expressing mesothelin. Cancer, 2020. 126(22): p. 4936-4947.
6. Adusumilli, P. S., et al., A Phase I Trial of Regional Mesothelin-Targeted CAR T-cell Therapy in Patients with Malignant Pleural Disease, in Combination with the Anti-PD-1 Agent Pembrolizumab. Cancer Discov, 2021. 11(11): p. 2748-2763.
7. Feng, Y., et al., A novel human monoclonal antibody that binds with high affinity to mesothelin-expressing cells and kills them by antibody-dependent cell-mediated cytotoxicity. Mol Cancer Ther, 2009. 8(5): p. 1113-8.
8. Ambrosetti, F., et al., proABC-2: PRediction of AntiBody contacts v2 and its application to information-driven docking. Bioinformatics, 2020. 36(20): p. 5107-5108.
9. Wang, C., et al., Design of a Novel Fab-Like Antibody Fragment with Enhanced Stability and Affinity for Clinical use. Small Methods, 2022. 6(2): p. e2100966.
10. Crooks, G. E., et al., WebLogo: a sequence logo generator. Genome Res, 2004. 14(6): p. 1188-90.
11. Santich, B. H., et al., Interdomain spacing and spatial configuration drive the potency of IgG-[L]-scFv T cell bispecific antibodies. Sci Transl Med, 2020. 12(534).
12. Nazarian, A. A., et al., Characterization of bispecific T-cell Engager (BiTE) antibodies with a high-capacity T-cell dependent cellular cytotoxicity (TDCC) assay. J Biomol Screen, 2015. 20(4): p. 519-27.
13. Liu, X., et al., Multiple proteases are involved in mesothelin shedding by cancer cells. Commun Biol, 2020. 3(1): p. 728.

We claim:

1. An isolated antibody, bispecific antibody or antigen-binding fragment thereof that binds to mesothelin, the antibody or antigen-binding fragment thereof comprising:
i. a heavy chain variable region comprising: a CDR1, a CDR2, and a CDR3 sequence from any of the sequences disclosed in Tables 2, 3, 4, or 5; and
a light chain variable region comprising: a CDR1, a CDR2, and a CDR3 sequence from any of the sequences disclosed in Tables 2, 3, 4, or 5.

2. The isolated antibody, bispecific antibody or antigen-binding fragment thereof of claim 1, comprising:
i. a heavy chain variable region comprising a sequence disclosed in Table 5; and
a light chain variable region comprising a sequence disclosed in Table 5.

3. The isolated antibody, bispecific antibody or antigen-binding fragment thereof of any one of claims 1-2 that further comprises a conjugated moiety.

4. The isolated antibody, bispecific antibody or antigen-binding fragment thereof of claim 3, wherein the conjugated moiety comprises a therapeutic agent, a solid support, an affinity agent, or a detectable label.

5. The isolated antibody, bispecific antibody or antigen-binding fragment thereof of any one of claims 3-4 wherein the conjugated moiety comprises an anti-cancer agent or a cytotoxin.

6. The isolated bispecific antibody or antigen-binding fragment thereof of any one of claims 1-5, further comprising an antibody or antigen-binding fragment thereof that specifically binds to an antigen that does not comprise a mesothelin epitope.

7. The bispecific antibody of claim 6, wherein the antibody or antigen-binding fragment thereof that specifically binds to an antigen that does not comprise a mesothelin epitope binds to an antigen comprising CD2, CD3, CD11a CD20, CD25 (IL2R), CD28, CD33, CD47, CD52, EGFR, VEGF, Integrin-alpha 3, GPIIb/IIIar, Protein F, TNF-alpha, TNF-beta, HER2/Neu, C5, or IgE.

8. A pharmaceutical composition comprising the isolated antibody, bispecific antibody or antigen-binding fragment thereof of any one of claims 1-7, and a pharmaceutically acceptable carrier.

9. A kit comprising the pharmaceutical composition of claim 8, or the antibody, bispecific antibody or antigen-binding fragment thereof of any one of claims 1-7, optional reagents, and instructions for use.

10. A method for treating cancer in a subject in need thereof, the method comprising administering to the subject an effective amount of an antibody, bispecific antibody or antigen-binding fragment thereof according to any one of claims 1-7, or the pharmaceutical composition according to claim 8.

11. The method according to claim 10, wherein the cancer comprises a solid tumor cell cancer.

12. An isolated polynucleotide encoding an antibody, bispecific antibody or antigen-binding fragment thereof according to any one of claims 1-7.

13. An isolated recombinant cell that produces an antibody, bispecific antibody or antigen-binding fragment thereof according to any one of claims 1-7.
